SEQUENCE LISTING (53 sequences)

SEQ ID NO: 1 - DNA, 1033 bp, Mus musculus
  CDS (48)...(1010)
cctgcctcagtgttggagctccccattgctgatgtgcaggcaagccgatgaaacgatggaaggacagaagaacaggcctgttgctgccattggtcctcctgttgtttggggcatgtagctcactggcatgggtatgtggccggcgaatgagtagcagatcccaacaacttaacaatgcttctgctatcgtggaaggcaaacctgcttctgctatcgtgggaggcaaacctgcaaacatcttggagttcccctggcatgtggggattatgaatcatggtagtcatctctgtgggggatctattctcaatgagtggtgggttctatctgcatcccattgcttcgaccaactaaacaactctaaattggagatcattcatggcactgaagacctcagcacaaagggcataaagtatcagaaagtggacaagttattcttgcacccaaagtttgatgactggctcctggacaacgacatagctttgctcttgctcaaatccccattaaacttgagtgtcaacaggatacctatctgcacttcagaaatctctgacatacaggcatggaggaactgctgggtgacaggatggggcattactaatactagtgaaaaaggagtccaacccacaattctgcaggcagtcaaagtggatctgtacagatgggattggtgtggctatattttgtctctattaaccaagaatatgctgtgtgctgggactcaagatcctgggaaggatgcctgccagggcgacagtggaggagctctcgtttgcaacaaaaagagaaacacagccatttggtaccaggtgggcattgtcagctggggcatgggctgtggcaagaagaatctgccaggagtatacaccaaggtgtcacactatgtgaggtggatcagcaagcagacagcgaaggcggggaggccttatatgtatgagcagaactctgcgtgccctttggtgctctcttgccgggctatcttgttcctatattttgtaatgtttcttctaacctgatgattaaacgtgagactgcc

SEQ ID NO: 2 - protein, 321 aa, Mus musculus
MKRWKDRRTGLLLPLVLLLFGACSSLAWVCGRRMSSRSQQLNNASAIVEGKPASAIVGGKPANILEFPWHVGIMNHGSHLCGGSILNEWWVLSASHCFDQLNNSKLEIIHGTEDLSTKGIKYQKVDKLFLHPKFDDWLLDNDIALLLLKSPLNLSVNRIPICTSEISDIQAWRNCWVTGWGITNTSEKGVQPTILQAVKVDLYRWDWCGYILSLLTKNMLCAGTQDPGKDACQGDSGGALVCNKKRNTAIWYQVGIVSWGMGCGKKNLPGVYTKVSHYVRWISKQTAKAGRPYMYEQNSACPLVLSCRAILFLYFVMFLLT

SEQ ID NO: 3 - DNA, 1034 bp, Mus musculus
  CDS (69)...(1025)
cccacgcgtncggttgtatcaatgtgggcagggcatcaaggcaggcaccactgcactggaatgacaacatgatgctcccacttctaattgcactgctcatggcttccaagggacaagctaaggaccagcaagaatcagttctgtgtggccacagacctgccttcccaaactcatcatggctgccattgcgggagctgcttgaggtccagcatggtgagttcccatggcaagtgagtatccagatgcttgggaaacacctgtgtggaggctccatcatccaccggtggtgggttctgacagcagcacactgcttcccgagaaccctattagaactggtagcagtcaatgtcactgtggtcatgggaatcaagactttcagtgacaccaacttagagagaaaacaagtgcagaagatcattgctcacagagactacaaaccgcccgaccttgacagcgacctctgcctgctcctacttgccacgccaatccaattcaataaagacaaaatgcccatctgcctgccacagagggagaactcctgggaccggtgctggatgtcagagtgggcatatactcatggccatggttcagccaaaggctcaaacatgcacctgaagaagctcagggtggttcagattagctggaggacatgtgcgaagagggtgactcagctctccaggaacatgctttgtgcttggaaggaagtgggcaccaacggcaagtgccagggagacagcggggcacccatggtctgtgctaactgggagactcggagactctttcaagtgggtgtcttcagctggggcataacttcaggatccagggggaggccaggcatttttgtgtctgtggctcagtttatcccatggatcctggaggagacacaaagggagggacgagcccttgccctctcaaaggcctcaaaaagtctcttggctggcagtccacgctaccatcccatattgctaagcatgggctctcaaatactgcttgctgccatattttctgatgataaatcaaattgctaagctctg

SEQ ID NO: 4 - protein, 319 aa, Mus musculus
MMLPLLIALLMASKGQAKDQQESVLCGHRPAFPNSSWLPLRELLEVQHGEFPWQVSIQMLGKHLCGGSIIHRWWVLTAAHCFPRTLLELVAVNVTVVMGIKTFSDTNLERKQVQKIIAHRDYKPPDLDSDLCLLLLATPIQFNKDKMPICLPQRENSWDRCWMSEWAYTHGHGSAKGSNMHLKKLRVVQISWRTCAKRVTQLSRNMLCAWKEVGTNGKCQGDSGAPMVCANWETRRLFQVGVFSWGITSGSRGRPGIFVSVAQFIPWILEETQREGRALALSKASKSLLAGSPRYHPILLSMGSQILLAAIFSDDKSNC

SEQ ID NO: 5 - DNA, 1035 bp, Homo sapiens
  CDS (73)...(867)
  misc_feature 1032: y=C or T/U
ctgtggctggcatgttgtcagctctggctggaggcaaaggtttggcaattttggactggaattgacaagaagatgttccagcttctaattcccctgcttttggcactcaagggacatgcccaggacaatccagaaaacgtacaatgtggccacaggcctgcttttccaaactcgtcatggttaccatttcatgaacggcttcaagtccagaatggtgagtgcccgtggcaagtgagtatccagatgtcacggaaacacctctgtggaggctcaatcttacattggtggtgggttctgacagccgcacactgcttccgaagaaccctattagacatggccgtggtaaatgtcactgtggtcatgggaacgagaacattcagcaacatccactcggagagaaagcaagtgcagaaggtcattattcacaaagattacaaaccgccccagctcgacagtgacctctctctgcttctacttgccacaccagtgcaattcagcaatttcaaaatgcctgtctgcctgcaggaggaggagaggacctgggactggtgttggatggcacagtgggtaacgaccaatgggtatgaccaatatgatgacttaaacatgcacctggaaaagctgagagtggtgcagattagccggaaagaatgtgccaagagggtaaaccagctgtccaggaacatgatttgtgcttcgaacgaaccaggcaccaatggtatcttcaagggagacagtggggcacctctggtttgtgctatttatggaacccagagactcttccaagtgggtgtcttcagtgggggcataagatctggctccagggggagacctggtatgtttgtgtctgtggctcaatttattccatgaagccaggaggagacagaaaaggaggggaaagcctacaccataatctcaggatccacgagaagccgagaagctcactggtgtgtgttcctcagtaccccttcttgctaggattggggtctcaaatgctgctggccaccatgtttaccggtgataaacctaacyrcw

SEQ ID NO: 6 - protein, 265 aa, Homo sapiens
MFQLLIPLLLALKGHAQDNPENVQCGHRPAFPNSSWLPFHERLQVQNGECPWQVSIQMSRKHLCGGSILHWWWVLTAAHCFRRTLLDMAVVNVTVVMGTRTFSNIHSERKQVQKVIIHKDYKPPQLDSDLSLLLLATPVQFSNFKMPVCLQEEERTWDWCWMAQWVTTNGYDQYDDLNMHLEKLRVVQISRKECAKRVNQLSRNMICASNEPGTNGIFKGDSGAPLVCAIYGTQRLFQVGVFSGGIRSGSRGRPGMFVSVAQFIP

SEQ ID NO: 7 - DNA, 1028 bp, Mus musculus
  CDS (38)...(1000)
gtcagcctggcctccaacacacagcacagccagagccatgatcctgccctccatcctgctacttgttgcccacaccctggaagcaaatgttgagtgtggtgtgagacccctgtatgatagcagaattcaatactccaggatcatagaagggcaggaggctgagctgggtgagtttccatggcaggtgagcattcaggaaagtgaccaccatttctgcggcggctccattctcagtgagtggtggatcctcaccgtggcccactgcttctatgctcaggagctttccccaacagatctcagagtcagagtgggaaccaatgacttaactacttcacccgtggaactagaggtcaccaccataatccggcacaaaggctttaaacggctgaacatggacaacgacattgccttgttgctgctagccaagcccttggcgttcaatgagctgacggtgcccatctgccttcctctctggcccgcccctcccagctggcacgaatgctgggtggcaggatggggcgtaaccaactcaactgacaaggaatctatgtcaacggatctgatgaaggtgcccatgcgtatcatagagtgggaggaatgcttacagatgtttcccagcctcaccacaaacatgctgtgtgcctcatatggtaatgagagctacgatgcttgccagggtgacagtgggggaccgcttgtctgcaccacagatcctggcagtaggtggtaccaggtgggcatcatcagctggggcaagagctgtggaaaaaaaggcttcccagggatatatactgtattggcaaagtataccctgtggattgagaaaatagcccagacagaggggaagcccctggattttagaggtcagagctcctctaacaagaagaaaaacagacagaacaatcagctctccaaatccccagccctgaactgcccccaaagctggctcctgccctgtctgctgtcctttgcactgcttagagccttgtccaactggaaataaaacaatgcagtctctgatccaccct

SEQ ID NO: 8 - protein, 321 aa, Mus musculus
MILPSILLLVAHTLEANVECGVRPLYDSRIQYSRIIEGQEAELGEFPWQVSIQESDHHFCGGSILSEWWILTVAHCFYAQELSPTDLRVRVGTNDLTTSPVELEVTTIIRHKGFKRLNMDNDIALLLLAKPLAFNELTVPICLPLWPAPPSWHECWVAGWGVTNSTDKESMSTDLMKVPMRIIEWEECLQMFPSLTTNMLCASYGNESYDACQGDSGGPLVCTTDPGSRWYQVGIISWGKSCGKKGFPGIYTVLAKYTLWIEKIAQTEGKPLDFRGQSSSNKKKNRQNNQLSKSPALNCPQSWLLPCLLSFALLRALSNWK

SEQ ID NO: 9 - DNA, 1123 bp, Homo sapiens
  CDS (41)...(1096)
ggcctctgtcacccccgggcccacagcacagcccagggccatgctcctgttctcagtgttgctgctcctgtccctggtcacgggaactcagctcggtccacggactcctctcccagaggctggagtggctatcctaggcagggctaggggagcccaccgccctcagccccgtcatccccccagcccagtcagtgaatgtggtgacagatctattttcgagggaagaactcggtattccagaatcacaggggggatggaggcggaggtgggtgagtttccgtggcaggtgagtattcaggcaagaagtgaacctttctgtggcggctccatcctcaacaagtggtggattctcactgcggctcactgcttatattccgaggagctgtttccagaagaactgagtgtcgtgctggggaccaacgacttaactagcccatccatggaaataaaggaggtcgccagcatcattcttcacaaagactttaagagagccaacatggacaatgacattgccttgctgctgctggcttcgcccatcaagctcgatgacctgaaggtgcccatctgcctccccacgcagcccggccctgccacatggcgcgaatgctgggtggcaggttggggccagaccaatgctgctgacaaaaactctgtgaaaacggatctgatgaaagcgccaatggtcatcatggactgggaggagtgttcaaagatgtttccaaaacttaccaaaaatatgctgtgtgccggatacaagaatgagagctatgatgcctgcaagggtgacagtggggggcctctggtctgcaccccagagcctggtgagaagtggtaccaggtgggcatcatcagctggggaaagagctgtggagataagaacaccccagggatatacacctcgttggtgaactacaacctctggatcgagaaagtgacccagctaggaggcaggcccttcaatgcagagaaaaggaggacttctgtcaaacagaaacctatgggctccccagtctcgggagtcccagagccaggcagccccagatcctggctcctgctctgtcccctgtcccatgtgttgttcagagctattttgtactgataataaaatagaggctattctttc

SEQ ID NO: 10 - protein, 352 aa, Homo sapiens
MLLFSVLLLLSLVTGTQLGPRTPLPEAGVAILGRARGAHRPQPRHPPSPVSECGDRSIFEGRTRYSRITGGMEAEVGEFPWQVSIQARSEPFCGGSILNKWWILTAAHCLYSEELFPEELSVVLGTNDLTSPSMEIKEVASIILHKDFKRANMDNDIALLLLASPIKLDDLKVPICLPTQPGPATWRECWVAGWGQTNAADKNSVKTDLMKAPMVIMDWEECSKMFPKLTKNMLCAGYKNESYDACKGDSGGPLVCTPEPGEKWYQVGIISWGKSCGDKNTPGIYTSLVNYNLWIEKVTQLGGRPFNAEKRRTSVKQKPMGSPVSGVPEPGSPRSWLLLCPLSHVLFRAILY

SEQ ID NO: 11 - DNA, 22 bp, Artificial Sequence
  “76A5sc2-B”,  an artificially synthesized primer sequence
gatcmacaggtgccagtcatca

SEQ ID NO: 12 - DNA, 20 bp, Artificial Sequence
  “SPORT SP6”,  an artificially synthesized primer sequence
atttaggtgacactatagaa

SEQ ID NO: 13 - DNA, 18 bp, Artificial Sequence
  “SPORT Fw”,  an artificially synthesized primer sequence
tgtaaaacgacggccagt

SEQ ID NO: 14 - DNA, 18 bp, Artificial Sequence
  “Sport RV”,  an artificially synthesized primer sequence
caggaaacagctatgacc

SEQ ID NO: 15 - DNA, 22 bp, Artificial Sequence
  “No9-C”,  an artificially synthesized primer sequence
atgcttctgctatcgtggaagg

SEQ ID NO: 16 - DNA, 20 bp, Artificial Sequence
  “SPORT T7”,  an artificially synthesized primer sequence
taatacgactcactataggg

SEQ ID NO: 17 - DNA, 22 bp, Artificial Sequence
  “No9-B”,  an artificially synthesized primer sequence
ctttgtgctgaggtcttcagtg

SEQ ID NO: 18 - DNA, 22 bp, Artificial Sequence
  “No9-G”,  an artificially synthesized primer sequence
cagtcaatgtcactgtggtcat

SEQ ID NO: 19 - DNA, 22 bp, Artificial Sequence
  “No9-J”,  an artificially synthesized primer sequence
acttgccgttggtgcccacttc

SEQ ID NO: 20 - DNA, 23 bp, Artificial Sequence
  “No9-P”,  an artificially synthesized primer sequence
gcactggaatgacaacatgatgc

SEQ ID NO: 21 - DNA, 22 bp, Artificial Sequence
  “No9-Q”,  an artificially synthesized primer sequence
attggcgtggcaagtaggagca

SEQ ID NO: 22 - DNA, 22 bp, Artificial Sequence
  “No9-N”,  an artificially synthesized primer sequence
cgagtctcccagttagcacaga

SEQ ID NO: 23 - DNA, 22 bp, Artificial Sequence
  “No9-M”,  an artificially synthesized primer sequence
cggtgacttggtcatgtctgtg

SEQ ID NO: 24 - DNA, 29 bp, Artificial Sequence
  “No9-K”,  an artificially synthesized primer sequence
ggatccatgaaacgatggaaggacagaag

SEQ ID NO: 25 - DNA, 22 bp, Artificial Sequence
  “No9-O”,  an artificially synthesized primer sequence
cgcagagttctgctcatacata

SEQ ID NO: 26 - DNA, 21 bp, Artificial Sequence
  “No9-A”,  an artificially synthesized primer sequence
ggcatgtagctcactggcatg

SEQ ID NO: 27 - DNA, 22 bp, Artificial Sequence
  “29 (-)”,  an artificially synthesized primer sequence
ggaccagcaagaatcagttctg

SEQ ID NO: 28 - DNA, 22 bp, Artificial Sequence
  “17  (+) 95 (+)”,  an artificially synthesized primer sequence
ctgctaccagttctaatttgcc

SEQ ID NO: 29 - DNA, 22 bp, Artificial Sequence
  “G3PDH 5′ ”,  an artificially synthesized primer sequence
gagattgttgccatcaacgacc

SEQ ID NO: 30 - DNA, 22 bp, Artificial Sequence
  “G3PDH 3′ ”,  an artificially synthesized primer sequence
gttgaagtcgcaggagacaacc

SEQ ID NO: 31 - DNA, 21 bp, Artificial Sequence
  “h-B”,  an artificially synthesized primer sequence
agaggtcactgtcgagctggg

SEQ ID NO: 32 - DNA, 22 bp, Artificial Sequence
  “h-D”,  an artificially synthesized primer sequence
tgtgaataatgaccttctgcac

SEQ ID NO: 33 - DNA, 22 bp, Artificial Sequence
  “h-A”,  an artificially synthesized primer sequence
ttcagcaacatccactcggaga

SEQ ID NO: 34 - DNA, 22 bp, Artificial Sequence
  “h-C”,  an artificially synthesized primer sequence
aagcaagtgcagaaggtcatta

SEQ ID NO: 35 - DNA, 22 bp, Artificial Sequence
  “h-F”,  an artificially synthesized primer sequence
cattggtcgttacccactgtgc

SEQ ID NO: 36 - DNA, 23 bp, Artificial Sequence
  “PRO1-E”,  an artificially synthesized primer sequence
attctcaatgagtggtgggttct

SEQ ID NO: 37 - DNA, 22 bp, Artificial Sequence
  “PRO1-D”,  an artificially synthesized primer sequence
ccagcacacagcatattcttgg

SEQ ID NO: 38 - DNA, 25 bp, Artificial Sequence
  “hPRO3-B”,  an artificially synthesized primer sequence
ggaaacagctcctcggaatataagc

SEQ ID NO: 39 - DNA, 25 bp, Artificial Sequence
  “hPRO3-D”,  an artificially synthesized primer sequence
tggatgggctagttaagtcgttggt

SEQ ID NO: 40 - DNA, 23 bp, Artificial Sequence
  “hPRO3-A”,  an artificially synthesized primer sequence
ttcgagggaagaactcggtattc

SEQ ID NO: 41 - DNA, 25 bp, Artificial Sequence
  “hPRO3-C”,  an artificially synthesized primer sequence
tgtgaaaacggatctgatgaaagcg

SEQ ID NO: 42 - DNA, 22 bp, Artificial Sequence
  “mPRO3-B”,  an artificially synthesized primer sequence
cacctactgccaggatctgtgg

SEQ ID NO: 43 - DNA, 25 bp, Artificial Sequence
  “mPRO3-D”,  an artificially synthesized primer sequence
ggctattttctcaatccacagggta

SEQ ID NO: 44 - DNA, 25 bp, Artificial Sequence
  “mPRO3-A”,  an artificially synthesized primer sequence
atagagtgggaggaatgcttacaga

SEQ ID NO: 45 - DNA, 21 bp, Artificial Sequence
  “mPRO3-C”,  an artificially synthesized primer sequence
gctacgatgcttgccagggtg

SEQ ID NO: 46 - protein, 12 aa, Mus musculus
GKCQGDSGAPMV

SEQ ID NO: 47 - protein, 12 aa, Artificial Sequence
  derived from Homo sapiens and Mus musculus
XXXXGXSGXXXX

SEQ ID NO: 48 - protein, 12 aa, Homo sapiens
GIFKGDSGAPLV

SEQ ID NO: 49 - protein, 6 aa, Artificial Sequence
  derived from Homo sapiens and Mus musculus
XXAXHC

SEQ ID NO: 50 - protein, 6 aa, Mus musculus
LTVAHC

SEQ ID NO: 51 - protein, 343 aa, Homo sapiens
MAQKGVLGPGQLGAVAILLYLGLLRSGTGAEGAEAPCGVAPQARITGGSSAVAGQWPWQVSITYEGVHVCGGSLVSEQWVLSAAHCFPSEHHKEAYEVKLGAHQLDSYSEDAKVSTLKDIIPHPSYLQEGSQGDIALLQLSRPITFSRYIRPICLPAANASFPNGLHCTVTGWGHVAPSVSLLTPKPLQQLEVPLISRETCNCLYNIDAKPEEPHFVQEDMVCAGYVEGGKDACQGDSGGPLSCPVEGLWYLTGIVSWGDACGARNRPGVYTLASSYASWIQSKVTELQPRVVPQTQESQPDSNLCGSHLAFSSAPAQGLLRPILFLPLGLALGLLSPWLSEH

SEQ ID NO: 52 - protein, 436 aa, Mus musculus
MVEMLPTVAVLVLAVSVVAKDNTTCDGPCGLRFRQNSQAGTRIVSGQSAQLGAWPWMVSLQIFTSHNSRRYHACGGSLLNSHWVLTAAHCFDNKKKVYDWRLVFGAQEIEYGRNKPVKEPQQERYVQKIVIHEKYNVVTEGNDIALLKITPPVTCGNFIGPCCLPHFKAGPPQIPHTCYVTGWGYIKEKAPRPSPVLMEARVDLIDLDLCNSTQWYNGRVTSTNVCAGYPEGKIDTCQGDSGGPLMCRDNVDSPFVVVGITSWGVGCARAKRPGVYTATWDYLDWIASKIGPNALHLIQPATPHPPTTRHPMVSFHPPSLRPPWYFQHLPSRPLYLRPLRPLLHRPSSTQTSSSLMPLLSPPTPAQPASFTIATQHMRHRTTLSFARRLQRLIEALKMRTYPMKHPSQYSGPRNYHYRFSTFEPLSNKPSEPFLHS

SEQ ID NO: 53 - protein, 246 aa, Mus musculus
MSALLILALVGAAVAFPVDDDDKIVGGYTCRESSVPYQVSLNAGYHFCGGSLINDQWVVSAAHCYKYRIQVRLGEHNINVLEGNEQFVDSAKIIRHPNYNSWTLDNDIMLIKLASPVTLNARVASVPLPSSCAPAGTQCLISGWGNTLSNGVNNPDLLQCVDAPVLPQADCEASYPGDITNNMICVGFLEGGKDSCQGDSGGPVVCNGELQGIVSWGYGCAQPDAPGVYTKVCNYVDWIQNTIADN

(19) United States
(12) Patent Application Publication
(10) Pub. No.: US 2009/0035799 A1
(43) Pub. Date: Feb. 5, 2009

(54) NOVEL TRYPSIN FAMILY SERINE PROTEASES

(76) Inventors: Chiaki SENOO, Niihari-gun (JP); Mariko Numata, Niihari-gun (JP)

Correspondence Address: CLARK & ELBING LLP, 101 FEDERAL STREET, BOSTON, MA, 02110, US

(21) Appl. No.: 12/206,511

(22) Filed: Sep. 8, 2008

Related U.S. Application Data
(62) Division of application No. 09/831,180, filed on Aug. 3, 2001.

(30) Foreign Application Priority Data
Nov. 4, 1998 (JP) 10/313366

(51) Int. Cl. C12Q 1/37; C07K 14/35; C12N 9/50; C07H 21/04; C12N 15/63; C07K 16/00; G01N 33/566; C12N 5/10; C12N 1/15; C12N 1/21; C12P 21/06

(52) U.S. Cl. 435/23; 530/350; 435/219; 536/23.5; 435/320.1; 435/325; 435/410; 435/254.1; 435/252.1; 435/69.1; 530/387.9; 436/501

(57) ABSTRACT

Two novel trypsin-family serine proteases specifically expressed in adult mouse testis (“Tespec PRO-1” and “Tespec PRO-2”), and a novel trypsin-family serine protease derived from mouse (“Tespec PRO-3”) have been isolated. Also, two novel trypsin-family serine proteases derived from human (“Tespec PRO-2” and “Tespec PRO-3”) have been isolated. It has been suggested that these proteins are involved in sperm differentiation and maturation, and sperm functions (e.g., fertilization). Therefore, these proteins are useful for development of novel therapeutics and diagnostics for infertility, as well as for development of novel contraceptives.

### CROSS-REFERENCE TO RELATED APPLICATIONS

This application is a divisional of, and claims priority from, U.S. application Ser. No. 09/831,180, filed May 3, 2001, which is the U.S. National Stage of International Application No. PCT/JP99/06111, filed Nov. 2, 1999, which, in turn, claims the benefit of Japanese application Ser. No. 10/313,366, filed Nov. 4, 1998, each of which is hereby incorporated by reference.

### TECHNICAL FIELD

The present invention relates to novel trypsin-family serine proteases, the genes encoding them, and the production and uses thereof.

### BACKGROUND ART

In the testis, the male reproductive organ, sperm, i.e. male gametes, are primarily formed through the following three-step process: (1) the self-reproduction of spermatogonium as the germ-line stem cell and the initiation of differentiation thereof to the sperm, (2) meiotic division of spermatocyte and the associated gene recombination, and (3) morphogenesis of the haploid spermatid to the sperm. The sperms formed in this manner are expelled into a female body by coitus, pass along the oviduct, and bind to an egg, the female gamete, to achieve fertilization (Yomogida, K. and Nishimune, Y. (1998) Protein, Nucleic acid and Enzyme, 511-521). To achieve fertilization, it is necessary for a sperm to move through the oviduct, adhere to and penetrate the zona pellucida on the egg surface, and then fuse with the egg.

A variety of proteases participate in these steps of the fertilization process. For example, an analysis using knockout mice (Krege, J. H. et al. (1995) Nature 375: 146-148; Esther Jr, C. R. et al. (1996) Lab. Invest. 74: 953-965) has revealed that sperm angiotensin-converting enzyme (testis ACE) plays an important role in the process of sperm transportation within the oviduct (Hagaman, J. R. et al. (1998) Proc. Natl. Acad. Sci. USA 95: 2552-2557). Fertilizing ability is markedly reduced in the male knockout mice that lack proprotein convertase 4 (PC4) (M. Mbikay et al. (1997) Proc. Natl. Acad. Sci. USA, 94: 6842-6846).

Regarding serine proteases, a variety of trypsin inhibitors inhibit in vitro fertilization, suggesting that trypsin-like serine proteases present in the sperm (the acrosome in particular) may digest the zona pellucida when the sperm penetrates the zona pellucida (Saling, P. M. (1981) Proc. Natl. Acad. Sci. USA, 78: 6231-6235; Benau, D. A. and Storey, B. T. (1987) Biol. Reprod., 36: 282-292; Liu D. Y. and Baker, H. W. (1993) Biol. Reprod., 48: 340-348). Previously, acrosin, a trypsin-family serine protease in the acrosome, was assumed to play this role (Brown, C. R. (1983) J. Reprod. Fertil., 69: 289-295; Kremling, H. et al. (1991) Genomics, 11: 828-834; Klemm, U. et al., (1990) Differentiation, 42: 160-166). However, acrosin knockout mice have been shown to have almost normal fertilizing ability, suggesting that other serine proteases which are present in the sperm, apart from acrosin, digest zona pellucida (Baba, T. et al. (1994) J. Biol. Chem., 269: 31845-31849; Adham, I. M. et al. (1997) Mol. Reprod. Dev., 46: 370-376). In ascidians, a trypsin-family serine protease, called spermosin, is expressed in the sperm (Sawada, H. et al. (1984) J. Biol. Chem., 259: 2900-2904). An antibody specific to this protease has been shown to inhibit fertilization in ascidians in a concentration-dependent manner (Sawada, H. et al., (1996) Biochem. Biophys. Res. Commun., 222: 499-504). Recently, cDNAs of the trypsin-family serine proteases, TESP1 and TESP2, which are expressed specifically in mouse acrosome, were cloned (Kohno, N. et al., (1998) Biochem. Biophys. Res. Commun., 245: 658-665). However, the roles these genes play in the fertilization process remains to be clarified. Moreover, serine proteases existing in the sperm and capable of digesting the zona pellucida have not yet been reported.

### DISCLOSURE OF THE INVENTION

An objective of the present invention is to provide novel trypsin-family serine proteases associated with spermatogenesis and sperm functions, the genes encoding these proteases and a production method and use thereof.

The present inventors attempted to amplify a gene designated as 76A5sc2 by polymerase chain reaction, and eventually found a gene fragment having a nucleotide sequence different from that of 76A5sc2 gene. Using this gene fragment, the present inventors have cloned the cDNAs containing entire open reading frames (ORF) of two novel trypsin-family serine proteases (“Tespec PRO-1” and “Tespec PRO-2”) expressed specifically in adult mouse testis. They have also analyzed the tissue-specific expression of these genes.

“Tespec PRO-1” (Testis specific expressed serine proteinase-1) is predicted to encode 321 amino acids. The deduced amino acid sequence contains trypsin-family serine protease motifs, “Trypsin-His” and “Trypsin-Ser” active sites, and exhibits significantly high homology to other trypsin-family serine proteases, such as acrosin, prostasin, trypsin and so on, in the regions of the two motifs and their neighboring regions. In the other regions, however, there are no known genes found to exhibit significant homology to this protein at the nucleotide or amino acid level. The foregoing demonstrates that this protein is a novel trypsin-family serine protease.

On the other hand, “Tespec PRO-2” is predicted to encode 319 amino acids. The protein has a “Trypsin-His” active site. With regard to the “Trypsin-Ser” active site, which consists of 12 amino acids, it is differs from that of the canonical motif by two amino acid residues. Such a difference is found in some other known trypsin-family serine proteases, and, thus, “Tespec PRO-2” is predicted to function as a protease. There are no known genes found to exhibit significant homology to “Tespec PRO-2” at the nucleotide and amino acid levels. Thus this protein is also a novel trypsin-family serine protease.

Interestingly, for “Tespec PRO-2”, a splicing isoform was found that comprises the first half region of “Tespec PRO-2” connected to the latter half region of “Tespec PRO-1”. This suggests that these two proteases are located very close to each other on the chromosome. Though a variety of splicing isoforms are found for “Tespec PRO-2”, these “Tespec PRO-2” isoforms do not retain a long stretch of ORF, and thus do not encode any proteases at all. The homology between “Tespec PRO-1” and “Tespec PRO-2” is 52.2% at the nucleotide level and 33.1% at the amino acid level.

The present inventors have also successfully cloned a cDNA for human “Tespec PRO-2” by RT-PCR and RACE, based on the nucleotide sequence of mouse “Tespec PRO-2”. Human “Tespec PRO-2” has been revealed to have 74.2% and 69.8% homology with mouse “Tespec PRO-2” at the nucleotide and amino acid levels, respectively. Further it has been clarified that human “Tespec PRO-2” is encoded on chromosome 8.

The present inventors have further succeeded in cloning a cDNA encoding human “Tespec PRO-3” by RT-PCR and RACE, based on the nucleotide sequence of mouse “Tespec PRO-1”. In addition, they also succeeded in cloning a cDNA that encodes mouse “Tespec PRO-3”, a mouse counterpart to human “Tespec PRO-3”.

Northern blot analysis using the coding region for “Tespec PRO-1” as a probe revealed that this gene is expressed merely in adult mouse testis, but it failed to identify the expression in other tissues or in the fetal stage. Likewise, RT-PCR analysis also showed that expression of “Tespec PRO-1” is distinctly high in the adult testis. In addition, “Tespec PRO-1” was verified to have increased expression in the testis of 18 day-old mice or older, but it was not expressed in the testis of 12 day-old mice or younger or in the spermatogenesis-defect mutant mice. Similar analysis was carried out for “Tespec PRO-2” and revealed that expression pattern of this gene is identical to that of “Tespec PRO-1”. These findings suggest that both “Tespec PRO-1” and “Tespec PRO-2” are involved in sperm differentiation and maturation, and/or sperm function (fertilization). It should be noted that trypsin-family serine proteases have been suggested to play important roles in fertilization.

Thus, the present inventors conclude that the proteins encoded by the isolated genes are likely serine proteases that play crucial roles in fertilization. Accordingly, they may be useful for developing new therapeutic or diagnostic agents for sterility, and/or for developing new contraceptives.

The present invention relates to novel trypsin-family serine proteases thought to be associated with spermatogenesis or sperm functions, the genes encoding them, production methods and the uses thereof. More specifically, the present invention provides:

1. a protein comprising the amino acid sequence selected from the group consisting of SEQ ID NO: 2, SEQ ID NO: 4, SEQ ID NO: 6, SEQ ID NO: 8, and SEQ ID NO: 10;

2. a protein functionally equivalent to the protein comprising an amino acid sequence selected from the group consisting of SEQ ID NO: 2, SEQ ID NO: 4, SEQ ID NO: 6, SEQ ID NO: 8, and SEQ ID NO: 10, wherein said protein is selected from the group of (a) and (b), wherein:

(a) is a protein comprising an amino acid sequence selected from the group consisting of SEQ ID NO: 2, SEQ ID NO: 4, SEQ ID NO: 6, SEQ ID NO: 8, and SEQ ID NO: 10, wherein one or more amino acids are deleted, added, inserted and/or substituted with different amino acids; and

(b) is a protein encoded by DNA that hybridizes to the DNA comprising the nucleotide sequence selected from the group consisting of SEQ ID NO: 1, SEQ ID NO: 3, SEQ ID NO: 5, SEQ ID NO: 7, and SEQ ID NO: 9;

3. a partial peptide of the protein according to any one of (1) and (2);

4. a fusion protein comprising the first protein according to any one of (1) and (2), fused with a second peptide;

5. a DNA molecule encoding the protein according to any one of (1) to (3);

6. a vector into which the DNA according to (5) is inserted;

7. a transformant having the DNA according to (5) in an expressible form;

8. a method for producing the protein according to any one of (1) to (3), said method comprising the steps of: culturing the transformant according to (7), and recovering the expressed protein from the transformant or the culture supernatant thereof;

9. a method of screening for a substrate of the protein according to any of (1) and (2), wherein the method comprises the following steps of:

(a) contacting a test sample with said protein;

(b) detecting the protease activity of said protein against the test sample; and

(c) selecting a compound that is digested or cleaved by said protease activity;

10. a substrate of the protein according to any of (1) and (2), wherein said substrate can be isolated by the method according to (9);

11. a method of screening for a compound capable of inhibiting the activity of the protein according to any of (1) and (2), said method comprising the following steps of:

(a) contacting the protein with the substrate of (10) in the presence of a test sample;

(b) detecting the protease activity of the protein against the substrate; and

(c) selecting a compound that reduces the protease activity relative to the protease activity detected in the absence of the test sample;

12. a compound that inhibits the activity of the protein according to any of (1) and (2), wherein said compound can be isolated by the method according to (11);

13. an antibody that binds to the protein according to any of (1) and (2);

14. a method for detecting or assaying the protein according to any of (1) and (2), said method comprising the steps of: contacting the antibody according to (13) with a test sample that is anticipated to contain the protein; and detecting or assaying formation of the immune-complex between the antibody and the protein; and

15. a nucleotide sequence specifically hybridizing to the DNA comprising the nucleotide sequence selected from the group consisting of SEQ ID NO: 1, SEQ ID NO: 3, SEQ ID NO: 5, SEQ ID NO: 7, and SEQ ID NO: 9, wherein the nucleotide sequence is at least 15 nucleotide in length.

The present invention provides novel trypsin-family serine proteases. Of the proteins provided in the present invention, the amino acid sequence of the mouse protein designated “Tespec PRO-1” is shown in SEQ ID NO: 2, the amino acid sequences of the mouse and human proteins designated “Tespec PRO-2” are shown in SEQ ID NO: 4 and SEQ ID NO: 6, respectively, and the amino acid sequences of the mouse and human proteins designated “Tespec PRO-3” are shown in SEQ ID NO: 8 and SEQ ID NO: 10, respectively. Nucleotide sequences of the cDNA encoding these proteins are shown in SEQ ID NO: 1, SEQ ID NO: 3, SEQ ID NO: 5, SEQ ID NO: 7, and SEQ ID NO: 9, respectively.

A high level of expression of the proteins of the present invention “Tespec PRO-1” and “Tespec PRO-2” were observed in the mouse testis (Examples 5 and 6). When these proteins are localized in the sperm, particularly in the acrosome region, they may function as key proteases for sperm to achieve fertilization by digesting the zona pellucida. Thus, the proteins of the present invention may be useful for developing new therapeutic and diagnostic agents for sterility or for developing new contraceptives.

The present invention also encompasses proteins that are functionally equivalent to mouse “Tespec PRO-1”, mouse “Tespec PRO-2”, human “Tespec PRO-2”, mouse “Tespec PRO-3”, or human “Tespec PRO-3” protein. As used herein, the term “functionally equivalent” refers to the retention of biological properties equivalent to mouse “Tespec PRO-1”, mouse “Tespec PRO-2”, human “Tespec PRO-2”, mouse “Tespec PRO-3”, or human “Tespec PRO-3” protein. Illustrative biological properties include, but are not limited to, for example, (i) trypsin-family serine protease activity as an activity property, (ii) trypsin-family serine protease motifs (“Trypsin-His” (PROSITE PS00134), “Trypsin-Ser” (PROSITE PS00135)) and/or similar sequences thereof, as well as significant homology to the amino acid sequence of mouse “Tespec PRO-1” protein, mouse “Tespec PRO-2” protein, human “Tespec PRO-2” protein, mouse “Tespec PRO-3” protein, or human “Tespec PRO-3” protein as the structural properties of the sequences (infra), and (iii) expression in the testis, as the expression property.

Methods for introducing mutations into the amino acid sequence of a protein, for example, may be used to obtain such functionally equivalent proteins. To obtain a protein into which mutations are introduced into its amino acid sequence, methods such as site-specific mutagenesis using synthetic oligonucleotide primers (Kramer, W. and Fritz, H. J. Methods in Enzymol., (1987) 154: 350-367), a PCR system for site-specific mutagenesis (GIBCO-BRL) and the Kunkel's method (Methods Enzymol., (1988) 85: 2763-2766) may be used. By these methods, a protein comprising the amino acid sequence of SEQ ID NO: 2, SEQ ID NO: 4, SEQ ID NO: 6, SEQ ID NO: 8, or SEQ ID NO: 10 can be modified to obtain a protein in which one or more amino acids in its amino acid sequence have been deleted, added, inserted and/or substituted with different amino acids without affecting the biological properties of the protein.

There is no particular limitation on the number of amino acids that may be mutagenized, as long as the protein retains the biological properties of the wild-type protein (comprising the amino acid sequence of SEQ ID NO: 2, SEQ ID NO: 4, SEQ ID NO: 6, SEQ ID NO: 8 or SEQ ID NO: 10). Such mutations include, but are not limited to, for example:
deletion of one or more amino acids, preferably, 2 to 30, and more preferably, 2 to 10 amino acids from any one of the amino acid sequences of SEQ ID NO: 2, SEQ ID NO: 4, SEQ ID NO: 6, SEQ ID NO: 8, and SEQ ID NO: 10;
addition of one or more amino acids, preferably, 2 to 30, and more preferably, 2 to 10 amino acids into any one of the amino acid sequences of SEQ ID NO: 2, SEQ ID NO: 4, SEQ ID NO: 6, SEQ ID NO: 8, and SEQ ID NO: 10; and
substitution of one or more, preferably, 2 to 30, and more preferably, 2 to 10 amino acids in any one of the amino acid sequences of SEQ ID NO: 2, SEQ ID NO: 4, SEQ ID NO: 6, SEQ ID NO: 8, and SEQ ID NO: 10, with different amino acids.

There is also no particular limitation on the amino acid sites for mutagenesis, so long as the protein retains the biological properties of the wild-type protein comprising any one of the amino acid sequences shown in SEQ ID NOs: 2, 4, 6, 8 and 10.

It is known that a protein comprising a modified amino acid sequence of another protein wherein one or more amino acid residues have been deleted, added, and/or substituted with different amino acids can maintain its biological activity (Mark, D. F. et al., Proc. Natl. Acad. Sci. USA, (1984) 81: 5662-5666; Zoller, M. J. & Smith, M., Nucleic Acids Research, (1982) 10: 6487-6500; Wang, A. et al., Science, 224: 1431-1433; Dalbadie-McFarland, G. et al., Proc. Natl. Acad. Sci. USA, (1982) 79: 6409-6413).

For example, proteins into which one or more amino acid residues have been added to proteins of the present invention include fusion proteins. A fusion protein is a protein made by fusing the protein of the present invention with another peptide. A fusion protein can be prepared in an artificial manner. For example, the DNA encoding the protein of the present invention can be ligated in-frame with a DNA encoding another peptide, and then introduced into an expression vector to express the fusion gene in a host using conventional methods. There is no particular restriction on the other peptides or proteins to be used for fusion with the protein of the present invention. Such peptides include, but are not limited to, for example, FLAG (Hopp, T. P. et al., BioTechnology, (1988) 6: 1204-1210), 6×His consisting of six histidine (His) residues, 10×His, influenza virus hemagglutinin (HA), human c-myc fragments, VSV-GP fragments, p18HIV fragments, T7-tag, HSV-tag, E-tag, SV40T antigen fragments, lck tag, α-tubulin fragments, B-tag, Protein C fragment, and other well-known peptides. Such proteins include, for example, GST (glutathione-S-transferase), HA (influenza virus hemagglutinin), immunoglobulin constant regions, β-galactosidase, MBP (maltose-binding protein), etc. Commercially available DNAs encoding these peptides or proteins may also be used to prepare fusion proteins.

Using well-known hybridization techniques (Sambrook, J et al., Molecular Cloning 2nd ed., 9.47-9.58, Cold Spring Harbor Lab. Press, 1989) and the DNA encoding the proteins of the present invention (DNA sequences of SEQ ID NOs: 1, 3, 5, 7 and 9) or a part thereof, one skilled in the art can isolate DNA homologous to the original DNA. Using the DNA thus obtained, one skilled in the art can routinely to obtain a protein functionally equivalent to the protein of the present invention. The present invention includes proteins that are functionally equivalent to the proteins of the present invention, including those which are encoded by DNA capable of hybridizing to the DNA encoding any of the aforementioned proteins of the present invention, or a part thereof, under a stringent condition. In the isolation of such hybridizable DNA from other organisms, there is no limitation on the type of organisms; such organisms include, but are not limited to, for example, human, mouse, rat, cattle, monkey, pig, etc. In the context of the present invention, the term “stringent conditions” typically refers to “42° C., 2×SSC, 0.1% SDS” and the like, preferably “50° C., 2×SSC, 0.1% SDS” and the like, and more preferably “65° C., 2×SSC, 0.1% SDS” and the like. Under these conditions, the higher the temperature is set, the higher the likelihood that DNA with higher homology will be obtained.

Proteins encoded by DNA isolated by the above hybridization techniques normally have high homology to the amino acid sequence of SEQ ID NO: 2, SEQ ID NO: 4, SEQ ID NO: 6, SEQ ID NO: 8, or SEQ ID NO: 10. In the context of the present invention, the term “high homology” typically refers to at least 60% homology, preferably at least 70% homology, more preferably at least 80% homology, even more preferably at least 95%. The degree of homology between two proteins can be determined using the algorithm described in Wilbur, W. J. and Lipman, D. J. Proc. Natl. Acad. Sci. USA, (1983) 80: 726-730.

The proteins of the present invention may differ in amino acid sequence, molecular weight, isoelectric point, presence or absence of a sugar chain, and form, according to the cells or hosts producing the proteins, or to the purification methods. However, as long as the obtained proteins retain the biological properties of the proteins comprising the amino acid sequence of SEQ ID NO: 2, SEQ ID NO: 4, SEQ ID NO: 6, SEQ ID NO: 8 or SEQ ID NO: 10, they are included in the present invention.

The protein of the present invention can be a naturally occurring protein or can be produced as a recombinant protein, utilizing a genetic recombination technique. A naturally occurring protein can be prepared, for example, by extracting proteins from tissue or cells (for example, testis) in which the proteins of the present invention are thought to be present, and then by performing affinity chromatography using the antibodies of the present invention described below.

Likewise, for example, to produce a recombinant protein, DNA encoding the protein of the present invention is incorporated into an expression vector in a manner such that the DNA is expressed under the control of expression regulatory regions, such as enhancers and promoters, and then transduced into host cells to express the protein.

Specifically, when mammalian cells are used, DNA corresponding to a conventional, useful promoter/enhancer, DNA encoding a protein of the present invention, and the poly A signal at the downstream region of the 3′ end of the coding region are functionally linked or constructed as a vector containing such DNA. Exemplary promoters/enhancers include, but are not limited to, human cytomegalovirus immediate early promoter/enhancer.

Other promoters/enhancers that can be used for protein expression include, but are not limited to, retroviral, polyomaviral, adenoviral and simian virus 40 (SV40) promoters/enhancers, and promoters/enhancers derived from mammalian cells, such as that of human elongation factor 1α (HEF1α).

This is easily carried out, for example, according to the method of Mulligan et al. (Nature (1979) 277: 108) when SV40 promoter/enhancer is used, and to the method of Mizushima et al. (Nucleic Acids Res. (1990) 18: 5322) when using HEF1α promoter/enhancer is used.

For a replication origin, those derived from SV40, polyomavirus, adenovirus, bovine papilomavirus (BPV), and the like may be used. To increase the copy number of the gene in the host cell, the expression vector may optionally contain a selectable marker, such as an aminoglycoside transferase (APH), thymidine kinase (TK), E. coli xanthine-guanine phosphoribosyl transferase (Ecogpt), or dihydrofolate reductase (dhfr) gene, etc.

When using E. coli, conventional useful promoters, a signal sequence for polypeptide secretion, and the gene to be expressed may be functionally linked to express the gene. Such promoters include, but are not limited to, for example, lacZ and araB promoters. When the lacZ promoter is used, the method of Ward et al. (Nature (1098) 341: 544-546; FASEB J. (1992) 6: 2422-2427) can be used. When the araB promoter is used, the method of Better et al. (Science (1988) 240: 1041-1043) may be followed.

To produce the protein into the periplasm of E. coli, the pelB signal sequence (Lei, S. P. et al., J. Bacteriol., (1987) 169: 4379) may be used as a signal for secretion of the protein.

Any expression vector can be used to produce the protein of the present invention so long as it is suitable for use with the present invention. Such expression vectors include, but are not limited to, for example, the adenoviral vector “pAdexLcw” and the retroviral vector “pZIPneo”. Also included are expression vectors derived from mammalians, including, but not limited to, for example, pEF and pCDM8; derived from insects, including, but not limited to, for example, pBacPAK8; derived from plants, including, but not limited to, for example, pMH1 and pMH2; derived from animal viruses, including, but not limited to, for example, pHSV, pMV, and pAdexLcw; derived from retroviruses, including, but not limited to, for example, pZIpneo; derived from yeast, including, but not limited to, for example, pNV11 and SP-Q01; derived from Bacillus subtilis, including, but not limited to, for example, pPL608 and pKTH50; and derived from E. coli, including, but not limited to, for example, pQE, pGEAPP, pGEMEAPP, pMALp2 and pREP4.

In the present invention, any production systems may be used to produce the protein. Such production systems for producing the protein include in vitro and in vivo production systems. Production systems using eukaryotic cells or prokaryotic cells may be used as in vitro production systems.

Among the production systems using eukaryotic cells are those using animal cells, plant cells, and fungal cells. Such animal cells include mammalian cells, such as CHO (J. Exp. Med. (1995) 108: 945), COS, myeloma, BHK (baby hamster kidney), HeLa, and Vero; amphibian cells, such as Xenopus oocytes (Valle, et al., Nature, (1981) 291: 358-340); insect cells, such as sf9, sf21 and Tn5. Particularly preferred are CHO cells, dhfr-CHO, a DHFR-deficient CHO cell (Proc. Natl. Acad. Sci. USA, (1980) 77: 4216-4220), and CHO K-1 (Proc. Natl. Acad. Sci. USA, (1968) 60: 1275).

Nicotiana tabacum-derived cells are plant cells that are well known for such use. They can be grown as callus culture. As such fungal cells, yeasts, such as the Saccharomyces genus, for example, Saccharomyces cerevisiae, filamentous bacteria such as the Aspergillus genus, for example, Aspergillus niger are known.

Among the production systems using prokaryotic cells is a production system using bacterial cells. Such bacterial cells include E. coli and Bacillus subtilis.

These cells are transformed with the DNA of interest, and the transformed cells are then cultured in vitro to obtain the proteins. The culture is performed according to conventional methods. For eukaryotic cells, culture media, such as DMEM, HEM, RPMI1640, and IMDM, can be used. These media may be used with a serum supplement, such as fetal calf serum (FCS), or used as a serum-free medium. Preferably pH of the culture ranges from about 6 to about 8. The culture is usually conducted for about 15 to 200 hours at a temperature of about 30° C. to 40° C., and, if necessary, the medium may be changed, aerated, and stirred.

On the other hand, in vivo production systems include systems using animals and plants. The DNA of interest is introduced into such a plant or animal, within which the protein is produced, and then the protein produced is recovered. As used herein, the term “host” encompasses such animals and plants as well.

The systems using animals include the production systems using mammals and insects. Such mammals include, but are not limited to, goats, pigs, sheep, mice, and cattle (Vicki Glaser, SPECTRUM Biotechnology Applications, 1993). When mammals are used, transgenic animals may be used. For example, the DNA of interest is inserted within a gene encoding a protein produced intrinsically in milk, such as goat β casein, to prepare a fusion gene. The DNA fragment containing the fusion gene in which the DNA of interest is inserted injected into a goat embryo, which is then introduced into a female goat. The protein is then collected from the milk produced from the transgenic goat, that which was born from the goat that had accepted the embryo, or descendents thereof. To increase the amount of the milk containing the protein that is produced from the transgenic goat, suitable hormone(s) may be given to the transgenic goats (Ebert, K. M. et al., Bio/Technology, (1994) 12: 699-702).

Silk worms are useful insects in the context of the present invention. When a silk worm is used, it is infected with a baculovirus into which the DNA of interest has been inserted, and the desired protein is obtained from the body fluids of the silk worm (Susumu, M. et al., Nature, (1985) 315: 592-594).

When a plant is used, tobacco, for example, can be used. When a tobacco plant is used, the DNA of interest is inserted into a plant expression vector, for example pMON 530, which is then introduced into a bacterium such as Agrobacterium tumefaciens. This bacterium is used to infect the tobacco plant, for example Nicotiana tabacum, to obtain the desired polypeptide from its leaves (Julian, K.-C. Ma, et al., Eur. J. Immunol., (1994) 24: 131-138).

The protein of the present invention thus obtained can be isolated from inside or outside of the cells, or from hosts and purified as a substantially pure and homogenous protein. The separation and purification of the protein is not limited to any particular method, and can be done using conventional methods for separation and purification. For example, chromatography columns, filtration, ultrafiltration, salting out, solvent precipitation, solvent extraction, distillation, immunoprecipitation, SDS-polyacrylamide gel electrophoresis, isoelectric focusing, dialysis, recrystallization and the like may be suitably selected or combined to separate/purify the protein.

Such chromatographies include, but are not limited to, for example, affinity chromatography, ion exchange chromatography, hydrophobic chromatography, gel filtration, reversed-phase chromatography, adsorption chromatography, etc. (Strategies for Protein Purification and Characterization: A Laboratory Course Manual. Ed Daniel R. Marshak et al., Cold Spring Harbor Laboratory Press, 1996). These chromatographies can be done by liquid chromatography, such as HPLC, FPLC, etc. The present invention encompasses the proteins highly purified by these purification methods.

Optionally, by treating with an appropriate modification enzyme before or after the proteins are purified, the proteins can be modified or their peptides can be partially removed. Such modification enzymes include, but are not limited to, trypsin, chymotrypsin, lysyl endopeptidase, protein kinase, and glucosidase.

The present invention also comprises partial peptides from the proteins of the present invention. Such peptides can be utilized, for example, as immunogens to give antibodies capable of binding to the proteins of the present invention. For this purpose, such peptides will contain at least 12 amino acid residues, and preferably, at least 20 amino acid residues. Partial peptides of the proteins of the present invention may be produced by genetic engineering techniques or using well-known methods for synthesizing peptides, or by cleaving the protein of the present invention with a suitable peptidase. To synthesize peptides, solid-phase synthesis and liquid-phase synthesis may be also used.

A protein of the present invention or a partial peptide thereof that is expressed in a host by using a genetic engineering technique can be isolated from the cells or extracellular materials and can be purified as a substantially pure and homogeneous protein. There is no limitation on the methods of isolation and purification of the protein; any of the generally used methods for protein purification may be used to isolate and purify the protein. Separation and purification of the protein can be achieved by properly selecting or combining methods including, but not limited to, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization.

Such chromatographies include, but not limited to, for example, affinity chromatography, ion exchange chromatography, hydrophobic chromatography, gel filtration, reversed-phase chromatography, adsorption chromatography, etc. (Strategies for Protein Purification and Characterization: A Laboratory Course Manual. Ed Daniel R. Marshak et al., Cold Spring Harbor Laboratory Press, 1996). These chromatographies can be done by liquid chromatography, such as HPLC, FPLC, etc. The present invention encompasses the proteins highly purified by these purification methods.

Optionally, by treating with an appropriate modification enzyme before or after the proteins are purified, the proteins can be modified or their peptides can be partially removed. Such modification enzymes include trypsin, chymotrypsin, lysyl endopeptidase, protein kinase, and glucosidase.

Further, the present invention provides for DNA encoding the proteins of the present invention mentioned above. The DNA of the present invention can be used not only to produce the proteins of the present invention in vivo and in vitro, but also for gene therapy of, for example, mammals (e.g., human). It is expected that the genes of the present invention, in particular, may be applied to the gene therapy of infertility. When used in the gene therapy, the DNA of the present invention is inserted into a vector and then administered to the target sites in the body. The method of administration may be ex vivo or in vivo. The vectors of the present invention include such vectors as used for gene therapy.

Genomic DNA or cDNA that encodes the protein of the present invention may be obtained by screening a genomic library, a cDNA library or the like, using a hybridization technique well known to one skilled in the art.

By using the obtained DNA or cDNA fragment as a probe, and further by screening genomic or cDNA libraries, the genes can be obtained from other cells, tissues, organs, or species. Genomic and cDNA libraries may be prepared by, for example, the method of Sambrook, J. et al., Molecular Cloning, Cold Spring Harbor Laboratory Press (1989). Also, commercially available DNA libraries may be used.

By determining the nucleotide sequence of the obtained cDNA, the translatable region encoded by the cDNA can be identified to obtain the amino acid sequence of the protein of the present invention.

Specifically, this can be done as follows. First, mRNA is isolated from cells, tissue, or an organ expressing a protein of the present invention. To isolate mRNA, a well-known method, for example, guanidine ultracentrifugation (Chirgwin, J. M. et al., Biochemistry, (1979) 18: 5294-5299), the AGPC method (Chomczynski, P. and Sacchi, N., Anal. Biochem., (1987) 162: 156-159), is used to isolate total RNA, from which mRNA is purified using mRNA Purification Kit (Pharmacia), etc. QuickPrep mRNA Purification Kit (Pharmacia) can be used to prepare mRNA directly.

cDNA is synthesized from the obtained mRNA by reverse transcriptase. It can be synthesized using the AMV Reverse Transcriptase First-strand cDNA Synthesis Kit (SEIKAGAKU KOGYO), etc. Also, it may be synthesized and amplified with the probes set forth herein, according to the 5′-RACE method (Frohman, M. A. et al., Proc. Natl. Acad. Sci. USA, (1988) 85: 8998-9002; Belyavsky, A. et al., Nucleic Acids Res., (1989) 17: 2919-2932) using the 5′-Ampli FINDER RACE KIT (Clontech) and the polymerase chain reaction (PCR).

The DNA fragment of interest is prepared from the PCR product obtained and ligated with vector DNA. Recombinant vectors are thus created, and they are introduced into host cells, such as E. coli. Colonies are selected to prepare the desired recombinant vector. The nucleotide sequence of the DNA of interest may be verified by a known method, for example, the dideoxy nucleotide chain termination method.

The DNA of the present invention can be designed to have a sequence with higher expression efficiency, taking into account the codon used in the host for the expression (Grantham, R. et al., Nucleic Acids Research, (1981) 9: r43-r74). Also, the DNA of the present invention may be modified using commercially available kits or well-known methods. Such modification(s) include, but are not limited to, for example, digestion with restriction enzymes, insertion of synthetic oligonucleotides or suitable DNA fragments, addition of linkers, insertion of a start codon (ATG) and/or stop codon (TAA, TGA, or TAG).

The DNA of the present invention encompasses, for example, the DNA comprising the nucleotide sequence extending from A at nucleotide 48 to C at nucleotide 1010 of the nucleotide sequence set forth in SEQ ID NO: 1; the DNA comprising the nucleotide sequence extending from A at nucleotide 69 to C at nucleotide 1025 of the nucleotide sequence set forth in SEQ ID NO: 3; the DNA comprising the nucleotide sequence extending from A at nucleotide 73 to A at nucleotide 867 of the nucleotide sequence set forth in SEQ ID NO: 5; the DNA comprising the nucleotide sequence extending from A at nucleotide 38 to A at nucleotide 1000 of the nucleotide sequence set forth in SEQ ID NO: 7; and the DNA comprising the nucleotide sequence extending from A at nucleotide 41 to C at nucleotide 1096 of the nucleotide sequence set forth in SEQ ID NO: 9.

The DNA of the present invention further encompasses DNA that hybridizes under stringent conditions to the DNA of any of the nucleotide sequences of SEQ ID NO: 1, SEQ ID NO: 3, SEQ ID NO: 5, SEQ ID NO: 7, and SEQ ID NO: 9, so long as the hybridizing DNA also encodes a protein functionally equivalent to the protein of the present invention.

The “stringent conditions” are typically “42° C., 2×SSC, 0.1% SDS” and the like, preferably “50° C., 2×SSC, 0.1% SDS” and the like, and more preferably “65° C., 2×SSC, 0.1% SDS” and the like. Under these conditions, the higher the temperature is set, the higher the likelihood that DNA with higher homology will be obtained.

The hybridizable DNA mentioned above may be, for example, naturally occurring DNA (for example, cDNA and genomic DNA). For naturally occurring DNA, organisms used for isolation of DNA encoding the functionally equivalent protein include, but are not limited to, for example, human, mouse, rat, cattle, monkey, pig, etc. For example, in such animals, in a working example described herein, the DNA of the present invention was isolated using cDNA derived from a tissue (for example, testis) in which mRNA capable of hybridizing to cDNA encoding the protein of the present invention was detected. DNA encoding the proteins of the present invention may be cDNA or genomic DNA, as well as synthetic DNA.

The present invention also provides for a method of screening for substrates of the proteins of the present invention. In the context of the present invention, the term “substrate” of the proteins of the present invention refers to a compound that is decomposed or cleaved at a specific site upon the binding of a protein of the present invention.

The compounds to be used as substrates are not restricted to proteins. For example, trypsin and chymotrypsin are known to cleave not only proteins but also amide and ester bonds in the derivatives of peptidic compounds (Farmer, D. A. et al., J. Biol. Chem., (1975) 250: 7366-7371; del Castillo, L. M. et al., Biochim. Biophys. Acta., (1971) 235: 358-69). Thus, in the present invention, there is no limitation on the types of substrates so long as they are decomposed or cleaved at a specific site upon the binding of a protein of the present invention. Such substrates may be peptides, analogues or derivatives (peptidic compounds) thereof, or non-peptidic compounds.

The method of screening for the substrates of the present invention comprises the steps of: (a) contacting a test sample with any of the protein of the present invention, (b) detecting the protease activity of the protein of the present invention against the test sample, and (c) selecting a compound that is decomposed or cleaved by the protease activity of the protein of the present invention.

Test samples used for screening are those expected to contain the substrates for the protein of the present invention, including, but not limited to, for example, cell extracts, extracts from animal tissues, expressed products of a gene library, purified or crude proteins, peptides, peptidic analogues or derivatives, non-peptidic compounds, synthetic compounds, and naturally occurring compounds.

In the screening of the substrates capable of binding to the proteins of the present invention, for example, a test sample is mixed with a protein of the present invention, and the mixture is incubated. Subsequently, a change within the test sample (cleavage or decomposition) is assayed. For example, when the test sample is a protein, the test sample can be assayed directly, or after azidated or bound to a fluorescent substance, to detect its changes in UV spectrum (Beynon, R. J. and Bond, J. S., Proteolytic enzymes (1989) IRL Press, pp. 25-55) and HPLC (Maier M, et al., FEBS Lett., (1988) 232: 395-398; Gau W, et al. Adv. Exp. Med. Biol. (1983) 156: 483-494) before and after the reaction, thereby measuring the protease activity.

When the test sample is a peptide (or an analogue or derivative thereof), such peptide (or an analogue or derivative thereof) consisting of several amino acids (often, but not limited to, one to five amino acid residues) is mixed with a protein of the present invention, and incubated. Subsequently, changes within the test sample are assayed. For example, the test sample may be labeled with a fluorescent compound (MEC: Kawabata S. et al. (1988) Eur. J. Biochem., 172: 17-25; AMC: Morita T. et al. (1977) J. Biochem., (Tokyo). 82: 1495-1498; AFC: Garrett J R, et al. (1985) Histochem. J., 17:805-817, etc.) at the carboxyl terminus. Then the protease activity may be assayed being indexed by the spectral changes of the fluorescent compound upon the cleavage of the test sample. Screening methods utilizing other fluorescently labeled peptide substrates can be used (Beynon, R. J. and Bond, J. S., Proteolytic enzymes (1989) IRL Press, pp. 25-55; Gossrau, R., et al. (1984) Adv. Exp. Med. Biol., 167: 191-207; and Yu, J. X. et al., J. Biol. Chem., (1994) 269: 18843-18848).

In addition, the principle of the above-mentioned methods can be applied to the screening by using, as the test compounds, synthetic compounds, a bank of naturally occurring substances, a lambda phage peptide display library, pin peptide synthetic compounds, etc. Also, high-throughput screening is possible by utilizing a combinatorial chemistry techniques (Wrighton, N. C., Farrell, F. X., Chang, R, Kashyap, A. K., Barbone, F. P., Mulcahy, L. S., Johnson, D. L., Barrett, R. W., Jolliffe, L. K., Dower, W. J., “Small peptides as potent mimetics of the protein hormone erythropoietin”, Science (UNITED STATES), Jul. 26, 1996, 273, p 458-64; Verdine, G. L., “The combinatorial chemistry of nature”, Nature (ENGLAND), Nov. 7, 1996, 384: 11-13; Hogan, J. C. Jr., “Directed combinatorial chemistry”, Nature (ENGLAND), Nov. 7, 1996, 384: 17-19).

Once substrates for the proteins of the present invention are isolated by using the screening method mentioned above, screening for inhibitors of the proteins of the present invention may then be conducted, the inhibitors being indexed by their inhibitory activity against the protease activity of the proteins of the present invention to the substrates. Thus the present invention also provides for a method of screening for compounds inhibiting the activity of the proteins of the present invention.

This method comprises the steps of: (a) contacting a protein of the present invention with its substrate in the presence of a test sample, (b) detecting protease activity of the protein of the present invention to the substrate, and (c) selecting a compound capable of lowering the protease activity relative to that detected in the absence of the test sample.

The proteins of the present invention useful for screening include authentic proteins, recombinant proteins, and partial peptides derived therefrom. Test samples useful for screening include, but are not limited to, cell culture supernatant, expression products of a gene library, peptides, peptide analogues or derivatives, purified or crude proteins (including antibodies), non-peptidic compounds, synthetic compounds, products from fermentation of microorganisms, extracts from marine organisms, plant extracts, cell extracts, extracts from animal tissues, etc.

Screening for inhibitors of the proteins of the present invention can be performed, for example, by using the systems as described in the following references (Beynon, R. J. and Bond, J. S., Proteolytic enzymes (1989), IRL Press, pp. 25-55; Maier, M. et al. (1988) FEBS Lett. 232: 395-398; Gau, W. et al. Adv. Exp. Med. Biol., (1983) 156: 483-494; Kawabata, S. et al. (1988) Eur. J. Biochem. 172: 17-25; Morita, T. et al. (1977) J. Biochem., (Tokyo) 82: 1495-1498; Garrett, J. R. et al. (1985) Histochem. J. 17: 805-817; Gossrau, R. et al. (1984) Adv. Exp. Med. Biol. 167: 191-207; Yu, J. X. et al., (1994) J. Biol. Chem., 269: 18843-18848). Further, given that a peptide substrate is a lead compound, compounds that have resulted from modification or substitution of a part of the structure of the lead compound can be used as the test compounds in the screening for inhibitors of the proteins of the present invention (Okamoto, S. et al. (1993) Methods Enzymol., 222: 328-340).

As described above, expression patterns and such of the proteins of the present invention suggest that the proteins of the present invention may be involved in sperm differentiation and maturation, or sperm function (fertilization). Inhibitors that are isolated using the screening method of the invention can be utilized to analyze the involvement of the proteins of the present invention in fertilization. For example, the inhibitors of the proteins of the present invention may be used for in vitro analysis of fertilization (Y. Toyoda et al., 1971, Jpn. J. Anim. Reprod., 16: 147-151; Y. Kuribayashi et al., 1996, Fertil. Steril., 66: 1012-1017), which can subsequently be used to determine whether the inhibitors are capable of inhibiting fertilization or not. Such an inhibitor of a protein of the present invention that is capable of inhibiting fertilization finds potential utility as, for example, a new contraceptive.

The compounds obtained by the screening method of the present invention may find practical utility as drugs for treating humans and other mammals, such as mice, rats, guinea pigs, rabbits, chicken, cats, dogs, sheep, pigs, cattle, monkeys, sacred baboons, and chimpanzees, according to a conventional means.

For example, the drugs can be administered orally, in the form of tablets coated with sugar, if necessary, capsules, elixirs or microcapsules, or they can be administered parenterally, in the form of injections of sterile solutions of water or other pharmaceutically acceptable solutions, or suspensions. For example, a compound having the activity to bind to a protein of the present invention can be mixed with a physiologically acceptable carrier, flavoring agent, excipient, vehicle, preservative, stabilizer, and/or bonding agent in the form of a unit dose that is required for pharmaceutical implementations accepted in general. These active ingredients enable the preparations to be obtained in a suitable volume within the indicated volume range.

Examples of additives that can be mixed into tablets and capsules include, but are not limited to, binders, such as gelatin, corn starch, tragacanth gum, and arabic gum; excipients, such as crystalline cellulose; swelling agents, such as cornstarch, gelatin, and alginic acid; lubricants such as magnesium stearate; sweeteners such as sucrose, lactose, and saccharin; and flavoring agents such as peppermint, Gaultheria adenothrix oil, and cherry. When the unit dosage form is a capsule, a liquid carrier, such as oil, can also be included in the above additives. Sterile compositions for injections can be formulated by following standard drug implementations provided for dissolving or suspending active substances in such a vehicle as distilled water, or natural vegetable oils, such as sesame oil and coconut oil.

For example, physiological saline and isotonic liquids including glucose or other adjuvants, such as D-sorbitol, D-mannose, D-mannitol, and sodium chloride, can be used as aqueous solutions for injections. These can be used in conjunction with suitable solubilizers, including, but not limited to, alcohol, specifically ethanol, polyalcohols such as propylene glycol and polyethylene glycol, non-ionic surfactants, such as Polysorbate 80™ and HCO-50.

Sesame oil or soybean oil can be used as an oleaginous liquid and may be used in conjunction with a solubilizer, such as benzyl benzoate and benzyl alcohol. In addition, such a liquid can be combined with a buffer, such as phosphate buffer and sodium acetate buffers; a pain-killer, such as benzalkonium chloride and procaine hydrochloride; a stabilizer, such as benzyl alcohol and phenol; and an anti-oxidant. The prepared injection is usually filled into a suitable ampoule.

Although the doses of the compounds that are obtained by the screening method of the present invention varies according to the symptoms, typically, an amount of about 0.1 to about 100 mg per day, preferably, about 1.0 to about 50 mg per day, and more preferably, about 1.0 to about 20 mg per day is administered orally to an adult (body weight 60 kg).

When administered parenterally, doses will differ, depending on the patient, target organ, symptoms and method of administration. The daily dose of, usually about 0.01 to about 30 mg, preferably about 0.1 to about 20 mg and more preferably about 0.1 to about 10 mg for an adult (body weight 60 kg) is advantageously administered by intravenous injection. For administration to other animals, the amount is converted to 60 kg of body-weight.

The present invention further provides antibodies capable of binding to a protein of the present invention. Such antibodies can be utilized for detection and purification of the protein of the present invention, as well as for in vitro analysis for fertilization. An antibody can be obtained as a monoclonal antibody or a polyclonal antibody by using a well-known method.

An antibody that specifically binds to a protein of the present invention can be prepared by using the protein of the present invention as a sensitizing antigen for immunization, according to a standard immunizing method, by fusing the immune cells obtained with any known parent cells, using a conventional method of cell fusion, and by screening for the cells producing an antibody, using a standard screening technique.

Specifically, a monoclonal or polyclonal antibody that specifically binds to the proteins of the present invention may be prepared as follows.

For example, the protein of the present invention that is used as a sensitizing antigen for obtaining the antibody is not restricted by the animal species from which it is derived, but is preferably a protein derived from mammals, for example, humans, mice, or rats, especially from humans. Proteins of human origin can be obtained based on the nucleotide sequence or amino acid sequence disclosed herein.

A protein to be used as a sensitizing antigen in the present invention may be a protein of the present invention or a partial peptide thereof. Partial peptides of a protein include, for example, amino (N) terminal fragments of the protein, and carboxyl (C) terminal fragments. In the context of the present invention, the term “antibody” of the present invention refers to an antibody that binds to the full-length protein or a fragment thereof.

A gene encoding a protein of the present invention or a fragment thereof is inserted into a well-known expression vector system, and the host cells described herein are transformed. Subsequently, the protein of interest or a fragment thereof is obtained from the host cells or the culture medium, using a well-known method, and used as a sensitizing antigen. Also, cells expressing the protein and lysate thereof, and a chemically synthesized protein of the present invention and a partial peptide thereof may be used as sensitizing antigens.

Mammals that can be immunized with the sensitizing antigens generally include, but are not limited to, Rodentia, Lagomorpha and Primates. To generate monoclonal antibodies, it is preferable to select a mammal by considering its compatibility with parent cells used for cell fusion.

Animals belonging to Rodentia include, but are not limited to, for example, mice, rats, hamsters, etc. Animals belonging to Lagomorpha include, but are not limited to, for example, rabbits, and Primates include, but are not limited to, for example, monkeys. Among monkeys, monkeys of the infraorder Catarrhini (Old World monkeys), for example, cynomolgus monkeys, rhesus monkeys, sacred baboons, chimpanzees, are used.

Any of a number of well-known methods may be used to immunize animals with a sensitizing antigen. For example, the sensitizing antigen is generally injected into mammals intraperitoneally or subcutaneously. Specifically, the sensitizing antigen is diluted or suspended with a buffer, such as physiological saline and phosphate-buffered saline (PBS), to be prepared in an appropriate amount, and, if desired, mixed with a suitable amount of a common adjuvant, such as Freund's complete adjuvant. The antigen thus prepared may be emulsified and then injected into the mammal. Thereafter, the sensitizing antigen suitably mixed with Freund's incomplete adjuvant is preferably challenged several times at four to 21 day intervals. A suitable carrier can also be used when an animal is immunized with the sensitizing antigen. After the immunization, elevation of the level of the desired antibody in the serum antibody is confirmed by a conventional method.

To obtain polyclonal antibodies against the proteins of the invention, blood is removed from the mammal sensitized with the antigen after the level of the desired antibody is confirmed to increase in the serum. Serum may be isolated from the blood by any well-known method. The serum containing the polyclonal antibody may be used as the polyclonal antibody, and further, if necessary, the fraction containing the polyclonal antibody may be isolated from the serum.

To obtain monoclonal antibodies, after verifying that the level of the desired antibody has been increased in the serum of the mammal sensitized with the above-described antigen, immunocytes are taken out from the mammal and used for cell fusion. In this procedure, preferable immunocytes for cell fusion are splenocytes in particular. Parent cells to be fused with the above immunocytes are preferably mammalian myeloma cells.

Cell fusion of the above immunocytes and myeloma cells may be routinely carried out using any well-known method, for example, the method of Milstein et al. (Galfre, G. and Milstein, C., Methods Enzymol., (1981) 73: 3-46).

Hybridomas obtained from the cell fusion are screened for selection by culturing them in a usual selective culture medium, for example, HAT culture medium (a medium containing hypoxanthine, aminopterin and thymidine). The culture in the HAT medium is continued for a sufficient period to eliminate the cells (non-fusion cells) except for the hybridomas of interest, usually for a few days to a few weeks. Subsequently, conventional limiting dilution analysis is performed to screen for and clone the hybridoma producing the antibody of interest.

In addition to obtaining the hybridomas mentioned above, by immunizing an animal other than human with the antigen, human lymphocytes, for example, human lymphocytes infected with EB virus, can be sensitized in vitro with a protein, protein-expressing cells or lysates thereof, and the sensitized lymphocytes can then be fused with myeloma cells derived from human that have the capacity of permanent cell division, for example U266, to obtain a hybridoma producing the human antibody of interest that comprises the binding activity to the protein (Unexamined Published Japanese Patent Application (JP-A) No. Sho 63-17688).

Moreover, a transgenic animal having a human antibody gene repertoire is immunized with an antigen, such as a protein, protein-expressing cells and cell lysate thereof to obtain antibody-producing cells, which are then fused with myeloma cells to obtain hybridomas. The hybridomas may be used to obtain a human antibody against the protein (WO92/03918, WO93/2227, WO94/02602, WO94/25585, WO96/33735, and WO96/34096).

Instead of producing antibodies from hybridomas, antibody-producing immunocytes such as sensitized lymphocytes that are immortalized with an oncogene may be used.

Such monoclonal antibodies, obtained as described above, can be produced as recombinant antibodies using genetic engineering techniques (for example, see Borrebaeck, C. A. K. and Larrick, J. W., THERAPEUTIC MONOCLONAL ANTIBODIES, Published in the United Kingdom by MACMILLAN PUBLISHERS LTD, 1990). A recombinant antibody may be produced as follows: the DNA encoding the antibody is cloned from a hybridoma or immunocytes, such as sensitized lymphocytes producing the antibody, and incorporated into a suitable vector, which is then introduced into a host to produce the antibody. The present invention encompasses such recombinant antibodies as well.

The antibody of the present invention may be an antibody fragment or a modified antibody, so long as it binds to a protein of the present invention. For example, antibody fragments include Fab, F(ab′)₂, Fv, or single chain Fv in which the H chain Fv and the L chain Fv are suitably linked via a linker (scFv, Huston, J. S. et al., Proc. Natl. Acad. Sci. USA, (1988) 85: 5879-5883). Specifically, antibody fragments can be produced by treating an antibody with an enzyme, for example, papain, pepsin, etc. Alternatively, a gene encoding any of the antibody fragments can be constructed, introduced into an expression vector, and then expressed in suitable host cells (for example, see Co, M. S. et al., J. Immunol., (1994) 152: 2968-2976; Better, M. and Horwitz, A. H., Methods Enzymol., (1989) 178: 476-496; Pluckthun, A. and Skerra, A., Methods Enzymol., (1989) 178: 497-515; Lamoyi, E., Methods Enzymol., (1986) 121: 652-663; Rousseaux, J. et al., Methods Enzymol., (1986) 121: 663-669; Bird, R. E. and Walker, B. W., Trends Biotechnol., (1991) 9: 132-137).

Any antibodies bound to various molecules, such as polyethylene glycol (PEG), can be used as modified antibodies. The “antibody” in the context of the present invention encompasses such modified antibodies as well. To obtain such a modified antibody, the antibody obtained may be chemically modified. These methods are well established in the art.

The antibody of the present invention may be obtained as a chimeric antibody, comprising a variable region derived from a non-human antibody and a constant region derived from a human antibody by using conventional techniques. Alternatively, the antibody of the present invention may be obtained as a humanized antibody, comprising a complementarity determining region (CDR) derived from a non-human antibody, a framework region (FR) derived from a human antibody, and a constant region.

Antibodies thus obtained can be purified to a homogenous state. The antibodies used in the present invention may be separated and purified by any conventional methods used for separation and purification of proteins. There is no limitation to such method at all. Concentration of the above mentioned antibodies can be determined by measuring absorbance, or by the enzyme-linked immunosorbent assay (ELISA), etc.

Assays for antigen-binding activity of the antibody of the present invention include, but are not limited to, ELISA, enzyme immunoassay (EIA), radio immunoassay (RIA), and immunofluorescence. For example, when ELISA is used, a protein of the present invention is placed in a plate coated with the antibody of the present invention, and subsequently, a sample containing the antibody of interest, for example, a culture supernatant of the cells producing the antibody or a purified antibody, is added to the plate. A secondary antibody that recognizes the antibody, labeled with an enzyme such as alkaline phosphatase, is added to the plate, which is then incubated and washed. Subsequently, an enzyme substrate, such as p-nitrophenyl phosphate, is added to the plate, and the antigen-binding activity is estimated by measuring the absorbance. As a protein, a fragment of the protein, such as a fragment comprising the C-terminal or N-terminal region, may be used. To evaluate the activity of the antibody of the present invention, BIAcore (Pharmacia) may be used.

By using these techniques, a method for detecting or determining the proteins of the present invention can be carried out, which method comprises the steps of contacting an antibody of the present invention with a sample presumed to contain a protein of the present invention and of detecting or determining the immune complex formed between the antibody and the protein. Since the method of the present invention for detecting or determining proteins can specifically detect or assay the proteins, it is useful in various experiments using proteins.

In addition, the present invention also provides nucleotides specifically hybridizing to the DNA of the nucleotide sequences shown in SEQ ID NOs: 1, 3, 5, 7 and 9, (or complementary DNA thereof), which nucleotides have a chain length of at least 15 nucleotides. As used herein, the term “specifically hybridizing” indicates that cross-hybridization does not significantly occur with DNA encoding other proteins under the usual hybridization conditions, preferably under stringent hybridization conditions. Such nucleotides are available as probes for detecting or isolating DNA that encodes a protein of the present invention, or as a primer for amplification. Taking the temperature for hybridization reaction, duration of the reaction, concentration of the probe or primer, length of the probe or primer, ionic strength, and others into account, those skilled in the art can properly select the stringency for the specific hybridization.

The mouse “Tespec PRO-1” and “Tespec PRO-2” genes of the present invention are specifically expressed in the testis. It is also believed that the genes are specifically expressed in mouse germ cells of 18 day old or older. Accordingly, these DNA can also be available as markers (diagnostics) for germ cells. In addition, since the genes of the present invention are thought to be involved in sperm differentiation and maturation, and/or sperm functions including the establishment of fertilization, these DNA are available for examination of infertility.

Further, “nucleotides specifically hybridizing to DNA comprising any one of the nucleotide sequences shown in SEQ ID NOs: 1, 3, 5, 7 and 9 (or complementary DNA thereof), which nucleotides have a chain length of at least 15 nucleotides” also include, for example, antisense oligonucleotides and ribozymes. An antisense oligonucleotide acts on a cell that produces a protein of the present invention to bind to DNA or mRNA encoding the protein, thereby inhibiting the transcription or translation, or enhancing degradation of the mRNA. Antisense oligonucleotides thus inhibit the expression of the proteins of the present invention, resulting in suppression of the functions of the proteins of the present invention. Such antisense oligonucleotides include, for example, an antisense oligonucleotide capable of hybridizing to a definite region of the nucleotide sequences shown in SEQ ID NOs: 1, 3, 5, 7 and 9. Such antisense oligonucleotides are preferably antisense oligonucleotides complementary to at least consecutive 15 nucleotides contained in any of the nucleotide sequences shown in SEQ ID NOs: 1, 3, 5, 7 and 9. More preferably, the above-mentioned antisense oligonucleotides have at least 15 continuous nucleotides containing the translation start codon.

Derivatives or modifications of the antisense oligonucleotides can also be used as antisense oligonucleotides. Such modifications include, but are not limited to, for example, lower alkyl phosphonate modifications, such as methyl-phosphonate or ethyl-phosphonate types; phosphorothioate modifications or phosphoroamidate-modifications, etc.

The antisense oligonucleotides include not only those having the nucleotides complementary to all the corresponding sequence of those constituting the given region of the DNA or mRNA, but also the oligonucleotides having one or more mismatches, as long as the DNA or mRNA and the oligonucleotides can selectively and stably hybridize with any of the nucleotide sequences of SEQ ID NOs: 1, 3, 5, 7 and 9. Such oligonucleotides are nucleotide sequence regions comprising at least 15 continuous nucleotides and exhibiting at least 70% homology, preferably at least 80% homology, more preferably at least 90% homology, most preferably at least 95% homology to the nucleotide sequence. The algorithm to determine the sequence homology mentioned in the references above.

The antisense oligonucleotides of the present invention can be made into an external preparation, such as a liniment or poultice, by mixing with a suitable base material which is inactive against the antisense oligonucleotides. Also, as needed, the antisense oligonucleotides can be formulated into tablets, powders, granules, capsules, liposome capsules, injections, solutions, nose-drops, and freeze-dried agents by adding excipients, isotonic agents, solubilizers, stabilizers, preservatives, pain-killers, etc. These can be prepared using the usual methods.

The antisense oligonucleotide derivatives of the present invention can be applied both in vivo and in vitro. They can be administered to the patient by directly applying onto the ailing site, or by injecting into a blood vessel and such, so that it will reach the ailing site. An antisense-mounting material can also be used to increase durability and membrane-permeability. Such materials include, but are not limited to, for example, liposome, poly-L lysine, lipid, cholesterol, lipofectin, and derivatives of these.

The dosage of the antisense oligonucleotide derivative of the present invention can be adjusted suitably according to the patient's condition and used in desired amounts. For example, a dose ranging from 0.1 to 100 mg/kg, preferably 0.1 to 50 mg/kg, can be administered.

### BRIEF DESCRIPTION OF THE DRAWINGS

FIG. 1 shows the mouse “Tespec PRO-1” cDNA sequence (SEQ ID NO:1) and the amino acid sequence thereof (SEQ ID NO:2). The active sites of trypsin-family serine protease are indicated by underlines. The poly A signal is marked with a wavy line.

FIG. 2 shows mouse “Tespec PRO-2” cDNA sequence (SEQ ID NO:3) and the amino acid sequence thereof (SEQ ID NO:4). The active sites of trypsin-family serine protease are indicated by underlines. The poly A signal is marked with a wavy line.

FIG. 3 shows an alignment of amino acid sequences of mouse “Tespec PRO-1” (SEQ ID NO:2), “Tespec PRO-2” (SEQ ID NO:4) and known proteases (SEQ ID NOS:51-53). Amino acids conserved among all the proteins are marked with “*” and amino acids with similar characteristics are marked with “.”. The active sites of trypsin-family serine protease are boxed.

FIG. 4 shows a result of amplification of the cDNA for mouse “Tespec PRO-1” and “Tespec PRO-2” by RT-PCR using mouse testis RNA. Positions of primers used are indicated in the top panel and the electrophoretic pattern of the products amplified by RT-PCR is indicated in the bottom panel.

FIG. 5 shows a schematic illustration indicating the structures of mouse “Tespec PRO-1” and “Tespec PRO-2” as well as splicing isoforms thereof. The numbers indicated below the boxes are the numbers of the nucleotides.

FIG. 6 shows tissue-specific expression of mouse “Tespec PRO-1” and “Tespec PRO-2” by RT-PCR. Positions of the primers used are indicated in the top panel and the electrophoretic pattern of the products amplified by RT-PCR is indicated in the bottom panel. 1; liver, 2; brain, 3; thymus, 4; heart, 5; lung, 6; spleen, 7; testis, 8; ovary, 9; kidney, 10; fetus of day 10-11, 11; distilled water (control).

FIG. 7 shows tissue-specific expression of mouse “Tespec PRO-1” and “Tespec PRO-2” investigated by Northern blotting. Positions of the primers used are indicated in the top panel and the result of the Northern blotting is indicated in the bottom panel. 1; 7-day-old embryo, 2; 11-day-old embryo, 3; 15-day-old embryo, 4; 17-day-old embryo, 5; heart, 6; brain, 7; spleen, 8; lung, 9; liver, 10; skeletal muscle, 11; kidney, 12; testis.

FIG. 8 shows the time of expression of mouse “Tespec PRO-1” and “Tespec PRO-2” in the testis by RT-PCR analysis. 1; W/Wv testis No. 1, 2; W/Wv testis No. 2, 3; W/Wv testis No. 3, 4; testis of 4 days after birth, 5; testis of 8 days after birth, 6; testis of 12 days after birth, 7; testis of 18 days after birth, 8; testis of 42 days after birth, 9; adult testis, 10; adult liver, 11; distilled water (control).

FIG. 9 shows the human “Tespec PRO-2” cDNA sequence (SEQ ID NO:5) and the amino acid sequence thereof (SEQ ID NO:6). The active sites of trypsin-family serine protease are indicated by underlines. The poly A signal is marked with a wavy line.

FIG. 10 shows a comparison of nucleotide sequence between mouse (SEQ ID NO:3) and human (SEQ ID NO:5) “Tespec PRO-2”. The nucleotides conserved between the two are boxed.

FIG. 11 shows a comparison of amino acid sequence between mouse (SEQ ID NO:4) and human (SEQ ID NO:6) “Tespec PRO-2”. Amino acid residues shared between the two are indicated by “*” and amino acid residues with similar characteristics are indicated by “.”. The active sites of trypsin-family serine protease are boxed.

FIG. 12 shows a result of PCR for chromosomal mapping of human “Tespec PRO-2”.

FIG. 13 shows the nucleotide (SEQ ID NO: 9) and amino acid (SEQ ID NO:10) sequences of human “Tespec PRO-3” cDNA. The active sites of trypsin-family serine protease are indicated by underlines. The poly A signal is marked with a wavy line.

FIG. 14 shows a comparison of nucleotide sequence homology in regard to “Tespec PRO-1” and “Tespec PRO-3”. Homologies of the nucleotide sequences are compared using full-length of mouse “Tespec PRO-1”, an about 400-bp region of EST from mouse “Tespec PRO-3”, and an about 200-bp region of human “Tespec PRO-3” obtained by RT-PCR under a low stringency condition as described in Example 9.

FIG. 15 shows the mouse “Tespec PRO-3” cDNA sequence (SEQ ID NO:7) and the amino acid sequence thereof (SEQ ID NO:8). The active sites of trypsin-family serine protease are indicated by underlines. The poly A signal is marked with a wavy line.

FIG. 16 shows a comparison of nucleotide sequence between mouse “Tespec PRO-3” (m. Tespec PRO-3) (SEQ ID NO:7) and human “Tespec PRO-3” (h. Tespec PRO-3) (SEQ ID NO:9). Nucleotides conserved between the two are boxed.

FIG. 17 shows a comparison of amino acid sequence between mouse “Tespec PRO-3” (m. Tespec PRO-3) (SEQ ID NO:8) and human “Tespec PRO-3” (h. Tespec PRO-3) (SEQ ID NO:10) Amino acid residues conserved between the two are boxed.

### BEST MODE FOR CARRYING OUT THE INVENTION

The present invention is illustrated more specifically below with reference to Examples, but is not to be construed as being limited to the examples described below.

### Example 1

### Isolation of “Tespec PRO-1” Gene Fragment

A mixture of plasmids derived from 5×10⁴ clones was isolated and purified from a plasmid library of mouse heart cDNA (GIBCO, 5×10⁹ cfu/ml). By using the plasmid mixture as a template, PCR amplification was performed according to the following procedure, using the primer “76A5sc2-B” specific to the gene that was named “76A5sc2” by the present inventors and the vector primer “SPORT RV”.

SuperScript Mouse heart cDNA library and SuperScript Mouse testis cDNA library (GIBCO, 5×10⁹ cfu/ml) were diluted 1:100. 1 μl aliquots of the diluted solutions were added to each of 16 tubes containing 3 ml of LB-Amp medium, and the mixtures were incubated at 30° C. Then the mixtures of plasmids were prepared with the QIAspin mini-prep kit (QIAGEN) (each plasmid preparation contains mixture of plasmids derived from 5×10⁴ independent clones). Using the plasmids from the mouse heart cDNA library as templates, PCR was carried out with Ampli Taq Gold (Perkin Elmer) as polymerase and the primer pair of 76A5sc2-B (SEQ ID NO: 11/5′-GAT CMA CAG GTG CCA GTC ATC A-3′) and SPORT SP6 (SEQ ID NO: 12/5′-ATT TAG GTG ACA CTA TAG AA-3′). The thermal cycling profile was: a pre-heat at 95° C. for 12 minutes, 40 cycles of denaturation at 96° C. for 20 seconds, annealing at 55° C. for 20 seconds and extension at 72° C. for 2 minutes, and subsequent final extension at 72° C. for 3 minutes.

The PCR reactions were subjected to electrophoresis on a 1.5% agarose gel. PCR products of about 0.7 Kb were cut out from the gel and then recovered by QIAquick Gel Extraction Kit (QIAGEN). The PCR products were cloned into pGEM T easy vectors (PROMEGA) by TA cloning using T4 DNA ligase (PROMEGA).

Eight colonies were selected from the colonies emerged, and the inserted fragments were amplified by colony PCR as follows.

The bacteria from each colony, which contain the recombinant gene, were directly suspended in 20 μl of PCR reaction solution containing a pair of the primers, SPORT FW (SEQ ID NO: 13/5′-TGT AAA ACG ACG GCC AGT-3′) and SPORT RV (SEQ ID NO: 14/5′-CAG GAA ACA GCT ATG ACC-3′), and KOD dash polymerase. PCR was performed by employing a thermal cycling profile of pre-heat at 94° C. for one minute, subsequently 32 cycles of denaturation at 96° C. for 15 seconds, annealing at 55° C. for 5 seconds, and extension at 72° C. for 25 seconds.

The amplification of the PCR products of interest was verified by agarose gel electrophoresis. If desired, the PCR products were purified by gel filtration with Microspin S-300 or S-400 (Pharmacia).

The PCR products from the above colony PCR or RT-PCR, were used as templates for sequencing. After the PCR reaction, the products generated were examined by agarose gel electrophoresis. If the products were contaminated, the PCR product of interest was cut out from the agarose gel to remove the contaminants. Otherwise, the products were purified by the above-mentioned gel filtration. Sequencing was performed by cycle sequence using Dye Terminator Cycle Sequencing FS Ready Reaction Kit, dRhodamine Terminator Cycle Sequencing FS ready Reaction Kit, or BigDye Terminator Cycle Sequencing FS ready Reaction Kit (Perkin-Elmer). Primers used were SPORT FW and SPORT RV. Unreacted primers, nucleotide monomers, and the like were removed by using a 96-well precipitation HL kit (AGTC). The nucleotide sequences were determined in the ABI 377 or ABI 377XL DNA Sequencer (Perkin-Elmer).

The result showed that seven plasmids contained the nucleotide sequence of 76A5sc2 and a single plasmid contained a distinct nucleotide sequence (the size of insert was about 0.5 Kb). This nucleotide sequence was then analyzed by searching the GCG database. Since this nucleotide sequence had an ORF, it was translated into an amino acid sequence. The amino acid sequence was also analyzed by searching the GCG database. The results showed that this gene fragment contained regions homologous to a number of known trypsin-family serine proteases at the nucleotide and amino acid levels. However, no known genes showed significant homology to this gene fragment over the entire regions, suggesting that this gene fragment has a novel origin. Further, the amino acid sequence was revealed to have a “Trypsin-His (PROSITE PS00134)” motif, one of the trypsin-family serine protease motifs. This also suggests that the gene fragment is derived from a novel protease gene.

### Example 2

### Cloning of Full-Length cDNA of the “Tespec PRO-1” Gene

By using the plasmid obtained from the SuperScript Mouse heart cDNA library in Example 1 as a template, plasmid library RACE was carried out employing Ampli Taq Gold as polymerase. The primer sets used in this experiment were a pair of No9-C (SEQ ID NO: 15/5′-ATG CTT CTG CTA TCG TGG AAG G-3′), which was newly designed based on the gene fragment isolated in Example 1, and a vector primer, SPORT FW or SPORT T7 (SEQ ID NO: 16/5′-TAA TAC GAC TCA CTA TAG GG-3′), and a pair of the primer No9-B (SEQ ID NO: 17/5′-CTT TGT GCT GAG GTC TTC AGT G-3′), which was newly designed based on the gene fragment and a vector primer, SPORT RV. The thermal cycling profile of the PCR was: a pre-heat at 95° C. for 12 minutes, 42 cycles of denaturation at 96° C. for 20 seconds, annealing at 55° C. for 20 seconds and extension at 72° C. for 5 minutes, and subsequent final extension at 72° C. for 3 minutes.

The PCR products were identified by agarose gel electrophoresis. Further, for these PCR products, the nucleotide sequences were determined directly or after cloned into pGEM T easy vector.

Since two PCR bands were obtained by 3′ RACE, the nucleotide sequences thereof were determined. The sequencing revealed that one of the two had the nucleotide sequence of the other in which a poly A stretch is attached to an internal site in the nucleotide sequence.

Likewise, 5′ RACE also gave two PCR bands with different sizes. DNAs from the respective bands were subcloned, and their nucleotide sequences were determined. The result revealed that the two were identical to each other in nucleotide sequence at the 3′ end, indicating that the two were different isoforms produced by alternative splicing.

The nucleotide sequences from the shorter band generated by 5′ RACE and the longer band generated by 3′ RACE were ligated to each other to give a nucleotide sequence encoding the entire protease, which was designated “Tespec PRO-1” (Testis specific expressed serine proteinase-1).

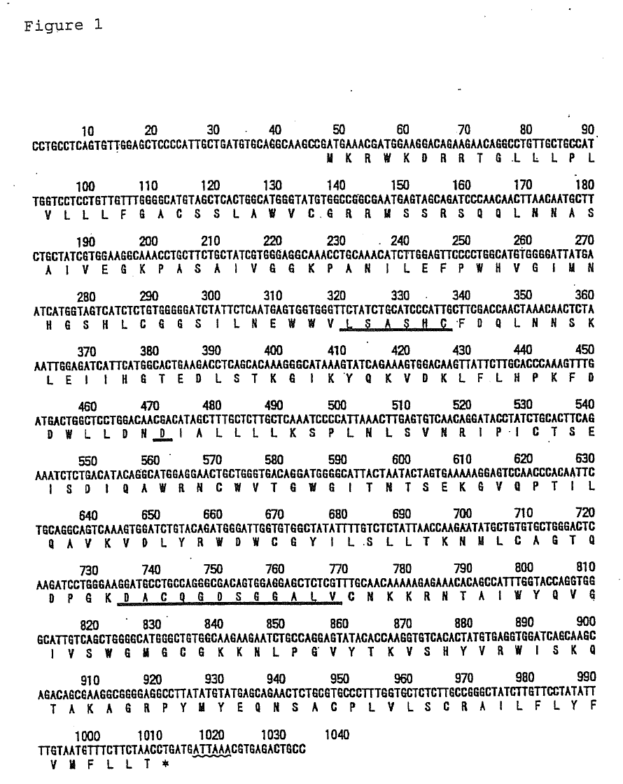

The resulting “Tespec PRO-1” cDNA contains 1033 nucleotides and is predicted to code for 321 amino acids (FIG. 1). The nucleotide sequence is shown in SEQ ID NO: 1 and the amino acid sequence is illustrated in SEQ ID NO: 2. The amino acid sequence contains a hydrophobic region at its N terminus, which is predicted to be a signal peptide. The amino acid sequence also has a region rich in hydrophobic amino acids at its C-terminus.

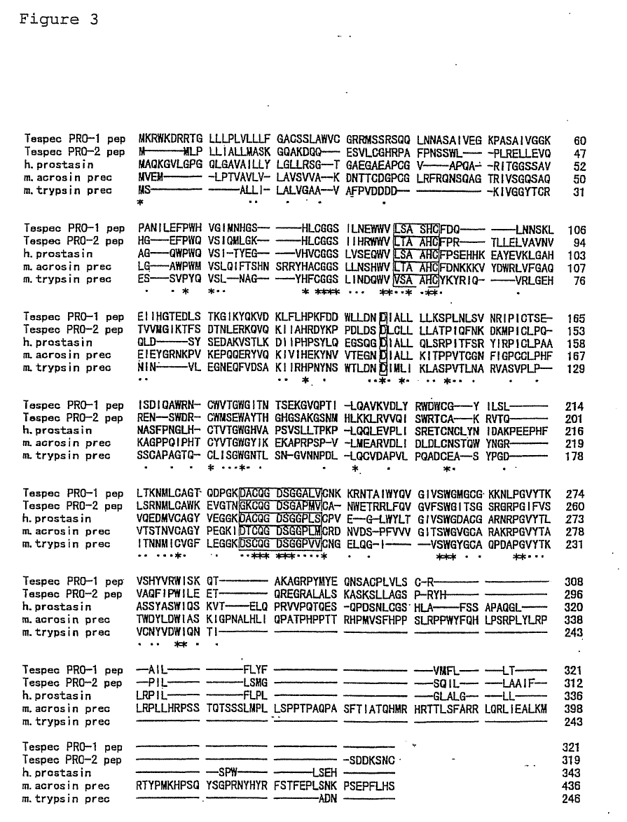

Based on the analytical search of the GCG, the amino acid sequence was proved to contain two types of trypsin-family serine protease motifs, “Trypsin-His (PROSITE PS00134)” and “Trypsin-Ser (PROSITE PS00135)”. PROSITE indicates “if a protein includes both the serine and histidine active site signatures, the probability of it being a trypsin family serine protease is 100%” (Brenner, S., 1988, Nature, 334: 528-530; Rawlings, N. D. and Barrett, A. J. (1994) Meth. Enzymol., 244: 19-61). “Tespec PRO-1” therefore can be regarded as a trypsin-family serine protease. The nucleotide sequence of this gene and its deduced amino acid sequence were analyzed by searching the GCG database. The results showed that the two motifs mentioned above and flanking region thereof exhibits high homologies to known trypsin-family serine proteases, such as acrosin, prostasin and trypsin. It was also revealed that the positions of aspartic acid residues required for the protease activity and the cysteine residues anticipated to be responsible for intramolecular disulfide bonding are well conserved relative to other proteases (FIG. 3). For the other region, however, no known genes or proteins were found to exhibit significant homology to this sequence at the nucleotide and amino acid levels, revealing that this protein is a novel trypsin-family serine protease.

### Example 3

### Cloning of Full-Length cDNA of the “Tespec PRO-2” Gene

For the band with larger molecular weight (the band with a nucleotide sequence different from that of “Tespec PRO-1” at the 5′ end), which was obtained during the cloning of “Tespec PRO-1” by 5′ RACE in Example 2, 3′ and 5′ RACE were carried out using newly synthesized primers designed based on the nucleotide sequence of “Tespec PRO-1” (No9-G or No9-J) as well as using, as templates, the plasmid mixture obtained from the SuperScript Mouse testis cDNA library in Example 1.

Specifically, PCR was conducted by using primer pairs of No9-G (SEQ ID NO: 18/5′-CAG TCA ATG TCA CTG TGG TCA T-3′) and SPORT FW, and No9-J (SEQ ID NO: 19/5′-ACT TGC CGT TGG TGC CCA CTT C-3′) and SPORT RV. In this PCR, Ampli Taq Gold was used as polymerase and its thermal cycling profile was as follows: a pre-heat at 95° C. for 12 minutes, 42 cycles of denaturation at 96° C. for 20 seconds, annealing at 55° C. for 20 seconds and extension at 72° C. for 5 minutes, and subsequent final extension at 72° C. for 3 minutes.

The nucleotide sequences of the PCR products were determined directly or after cloned into pGEM T easy vector.

Two 3′ RACE products were obtained by 3′ RACE, both of which were sequenced. By this analysis, the two nucleotide sequences were showed to have an identical region at their 5′ ends but distinct regions at their 3′ ends. One of the sequences was identical to the aforementioned nucleotide sequence having the sequence of “Tespec PRO-1” in which a poly A stretch is attached to an internal site of the sequence. The other sequence contained a nucleotide sequence different from that of “Tespec PRO-1” at its 3′ end.

Multiple bands were given by 5′ RACE. Those bands were subcloned, and their nucleotide sequences were determined. The result showed that all these bands shares an identical 3′ terminal sequence. Thus they are shown to be splicing isoforms. Since one of the 5′ RACE products has a long ORF, the 5′ RACE product and the above-mentioned 3′RACE product whose nucleotide sequence is different from that of “Tespec PRO-1” at the 3′ end were assembled together, thereby giving a nucleotide sequence presumed to encode a protease. This sequence was named “Tespec PRO-2”. The nucleotide sequence is shown in SEQ ID NO: 3, and the deduced amino acid sequence is indicated in SEQ ID NO: 4.

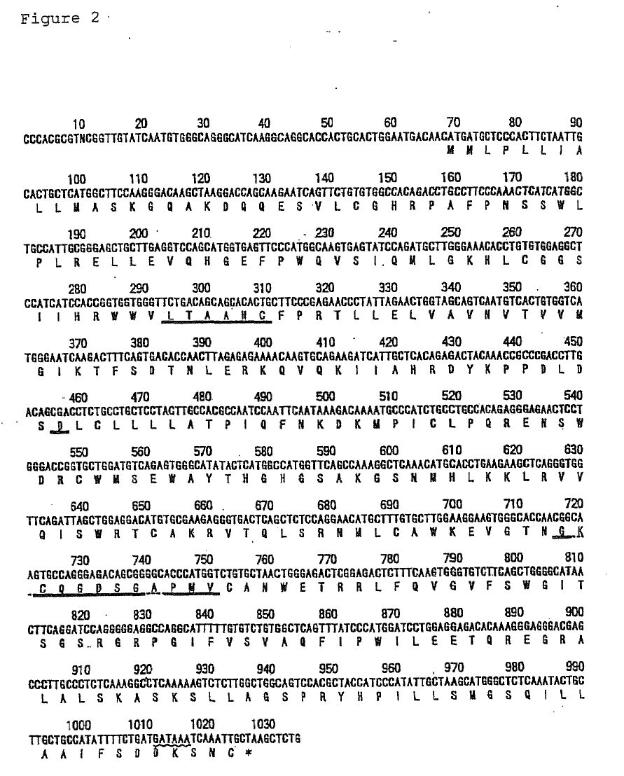

“Tespec PRO-2” cDNA thus obtained consists of 1034 nucleotides (FIG. 2) and its 5′ non-coding region consists of 68 nucleotides. By contrast, the 3′-non-coding region of this cDNA is very shorter, consisting of only nine nucleotides. A putative poly A signal found in this cDNA is GATAAA, and it is predicted to be weaker signal as compared to the signal generally recognized in mRNAs (AAUAAA). Based on the sequence of this cDNA, “Tespec PRO-2” is predicted to encode 319 amino acids, which contains a possible region of signal peptide at its N-terminus. But, unlike “Tespec PRO-1”, the protein does not contain a region rich in hydrophobic amino acids at its C-terminus. While the amino acid sequence contains a trypsin-family serine protease motif, “Trypsin-His”, the “Trypsin-Ser” motif of this protein (GKCQGDSGAPMV) (SEQ ID NO:46) contains 2 amino acid residues that are deviated from the consensus sequence of the motif that consists of 12 amino acid residues ([DNSTAGC]-[GSTAPIMVQH]-X-X-G-[DE]-S-G-[GS]-[SAPHV]-[LIVMFYWH]-[LIVMFYSTANQH]) (SEQ ID NO:47). However, some known trypsin-family serine proteases have sequences that are different from the consensus sequence at several amino acid residues. “Tespec PRO-2” obtained is predicted to function as a protease.

The nucleotide sequence of “Tespec PRO-2” and its deduced amino acid sequence were analyzed by searching the GCG database. The results showed that, like “Tespec PRO-1”, the two motifs of “Tespec PRO-2” mentioned above and flanking region thereof exhibits high homologies to known trypsin-family serine proteases. It was also revealed that the positions of aspartic acid residues required for the protease activity and the cysteine residues anticipated to be responsible for intramolecular disulfide bonding are highly conserved relative to other proteases (FIG. 3). For the other region, however, no known genes or proteins were found to exhibit significant homology at the nucleotide and amino acid levels, revealing that this protein is a novel trypsin-family serine protease.

### Example 4

### Splicing-Isoforms of “Tespec PRO-1” and “Tespec PRO-2”

Homologies between “Tespec PRO-1” and “Tespec PRO-2” were 52.2% and 33.1% at the nucleotide and amino acid levels, respectively. These values are of similar extent, compared to those of other known trypsin-family serine proteases.

The splicing isoform of “Tespec PRO-2” obtained by 5′ RACE in Example 3 does not appear to encode a protease, since it contains multiple termination codons in the nucleotide sequence at the splicing junction and in the region that is missing in “Tespec PRO-2”, which will prevent ORF extending. The splicing isoform was analyzed in more detail by RT-PCR as follows.

Based on the nucleotide sequence obtained by cDNA cloning, primers were synthesized which include No9-P (SEQ ID NO: 20/5′-GCA CTG GAA TGA CAA CAT GAT GC-3′), No9-Q (SEQ ID NO:21/5′-ATT GGC GTG GCA AGT AGG AGC A-3′), No9-N (SEQ ID NO: 22/5′-CGA GTC TCC CAG TTA GCA CAG A-3′), No9-M′ (SEQ ID NO: 23/5′-CGG TGA CTT GGT CAT GTC TGT G-3′), No9-K (SEQ ID NO: 24/5′-GGA TCC ATG AAA CGA TGG AAG GAC AGA AG-3′), No9-G, No9-J, and No9-O (SEQ ID NO: 25/5′-CGC AGA GTT CTG CTC ATA CAT A-3′). RT-PCR was performed by using these primers, cDNAs prepared from mouse tissue as templates, Ampli Taq Gold as polymerase and the thermal cycling profile of: pre-heating at 95° C. for 12 minutes, 40 cycles of denaturation at 96° C. for 20 seconds, annealing at 60° C. for 20 seconds and extension at 72° C. for 1 minute, and subsequent final extension at 72° C. for 3 minutes. PCR reactions were subjected to electrophoresis on a 1.5% Seakem GTG agarose (TaKaRa).

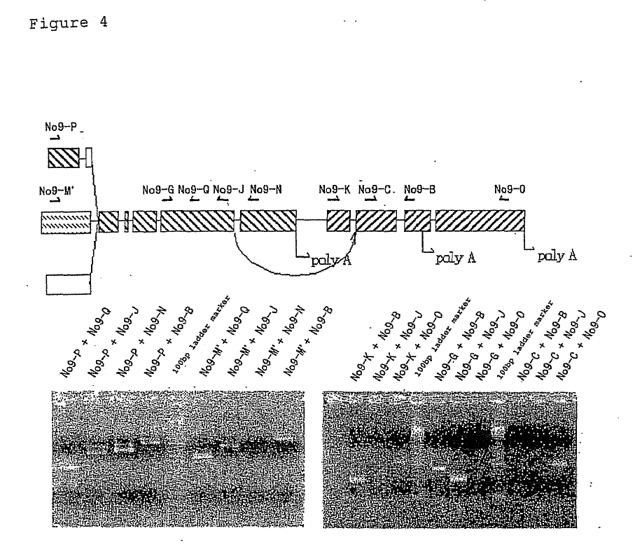

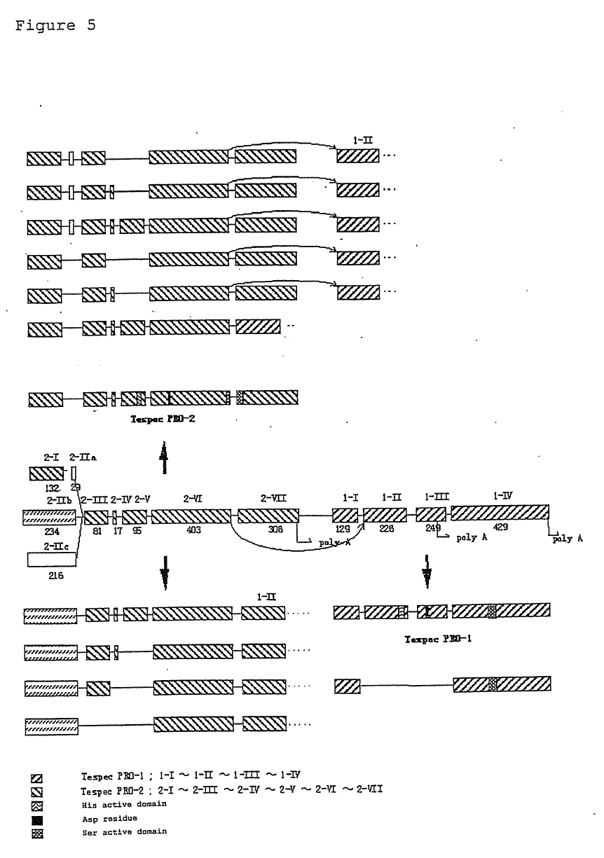

The results of RT-PCR analysis (FIGS. 4 and 5) showed that isoforms having the boxes (2-I)-(2-III)-(2-VI) at the 5′ end were appear to be dominant in the population of the splicing isoforms of “Tespec PRO-2”. The population appears to be larger than that of “Tespec PRO-2”. The RT-PCR analysis has verified cDNA isoforms with Box 2-I in which the Box is connected via Box 2-VI to Box 2-VII or Box 1-II (the latter is suspected to be a chimeric cDNA molecule with “Tespec PRO-1”). In contrast, the analysis also revealed that there is only a single type of cDNA isoform with Box 2-IIb, a chimeric cDNA with “Tespec PRO-1” in which the Box is connected via Box 2-VI to Box 1-II (FIGS. 4 and 5). Such chimeras may be formed because “Tespec PRO-2” and “Tespec PRO-1” are located in the close proximity on the chromosome, as well as due to weak signal intensity of the poly A signal in “Tespec PRO-2”. It remains to be clarified why such splicing isoforms (encoding only short proteins) that are seemingly meaningless exist. However, there is a possibility that the expression of “Tespec PRO-2” is regulated by splicing as well as transcriptionally.

### Example 5

### Tissue Distribution of the “Tespec PRO-1” and “Tespec PRO-2” Genes

Tissue distribution of “Tespec PRO-1” and “Tespec PRO-2” were investigated by RT-PCR. Total RNAs (Ambion) isolated from 10 types of adult mouse tissue (liver, brain, thymus, heart, lung, spleen, testis, uterus, kidney, and fetus of day 10-11) were used to synthesize cDNA by reverse transcription using SuperScript II (GIBCO) as a reverse transcriptase and using (dT)₃₀VN primer. The resulting cDNAs were used as templates for RT-PCR. QUICK-Clone cDNA from mouse 7-day embryo as well as 17-day embryo (CLONTECH) was also used as a template for RT-PCR.

“Tespec PRO-1”-specific primers used were No9-A (SEQ ID NO: 26/5′-GGCATGTAG CTC ACT GGCATG-3′) and No9-B. “Tespec PRO-2”-specific primers used were 29(−) (SEQ ID NO: 27/5′-GGA CCA GCA AGA ATC AGT TCT G-3′) and 17(+)₉₅(+) (SEQ ID NO: 28/5′-CTG CTA CCA GTT CTA ATT TGC C-3′) G3PDH control primers used were G3PDH 5′ (SEQ ID NO: 29/5′-GAG ATT GTT GCC ATC AAC GAC C-3′) and G3PDH 31 (SEQ ID NO: 30/5′-GTT GAA GTC GCA GGA GAC AAC C-3′). Polymerase used was Ampli Taq Gold and the thermal cycling profile of PCR was: pre-heat at 95° C. for 12 minutes, 42 cycles of denaturation at 96° C. for 20 seconds, annealing at 60° C. for 20 seconds and extension at 72° C. for 30 seconds (28 cycles for G3PDH), and subsequent final extension at 72° C. for 3 minutes. The PCR reactions were subjected to electrophoresis on a 1.5% Seakem GTG agarose (TaKaRa).

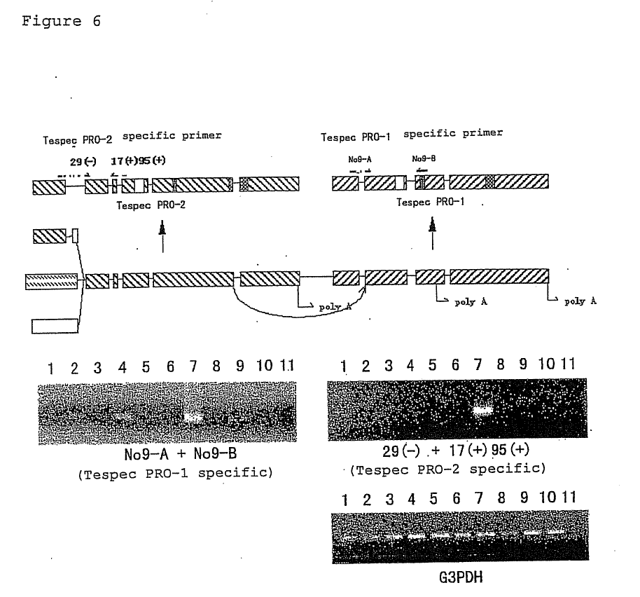

The result showed that both “Tespec PRO-1” and “Tespec PRO-2” were expressed in the testis at high levels (FIG. 6). Interestingly, it was also shown that these genes, despite of being cloned from the plasmid library of mouse heart cDNA, were hardly expressed in the heart. In the tissue other than the testis, the bands of interest were observed, though they were very faint.

In addition, tissue distribution was analyzed by mouse MTN blot (CLONTECH), using, as probes, a part of the coding region of “Tespec PRO-1” (the region containing the entire sequence of Box 1-II; the nucleotide positions 110 to 401) and a region in the vicinity of exon 2-VI of “Tespec PRO-2” (nucleotide positions 340 to 723) (this probe may be recognize “Tespec PRO-2” and all the splicing isoforms thereof, since it covers the region that is common to many of the splicing isoforms of “Tespec PRO-2”, therefore it is not a “Tespec PRO-2”-specific probe).

The RT-PCR products amplified by using cDNAs from adult mouse testis as templates and No9-A and No9-B primers were labeled with [α-³²P] dCTP by using the Megaprime DNA labeling system (Amersherm), and unreacted [α-³²P] dCTP was removed to give the “Tespec PRO-1” probe. Likewise, the “Tespec PRO-2” probe was prepared by PCR using No9-G and No9-J primers and subsequently by labeling with [α-³²P] dCTP. The hybridization was carried out at 68° C. by using Mouse Multiple Tissue Northern (MTN) blot and Mouse Embryo Multiple Tissue Northern (MTN) blot (CLONTECH) in ExpressHyb Hybridization Solution (CLONTECH), according to the manufacturer's instruction.

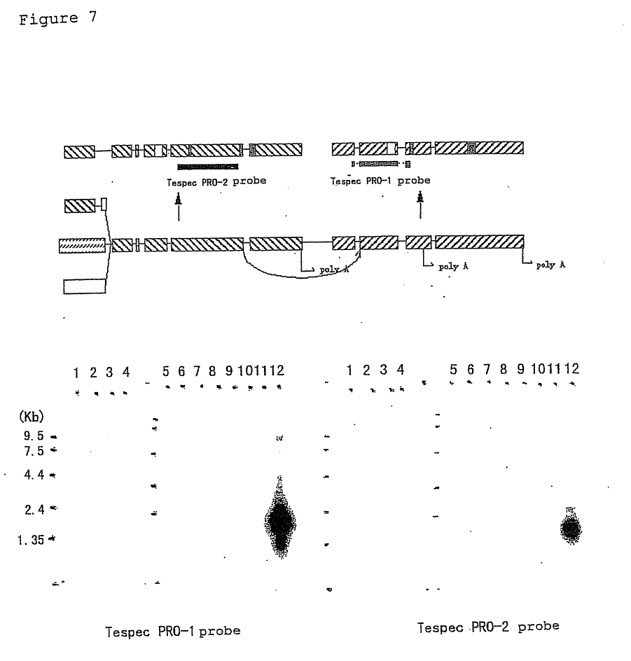

A band about 1.2 Kb in length was observed only in the testis by using the “Tespec PRO-1” probe (FIG. 7). This band was not detected in the tissue other than the testis, as well as in the fetus. Like the “Tespec PRO-1” probe, the “Tespec PRO-2” probe also detected an about 1.2-Kb band only in the testis (FIG. 7). The band was not detected in tissue other than the testis, as well as in the fetus.

The results described above demonstrate that both “Tespec PRO-1” and “Tespec PRO-2” are specifically expressed in the testis.

### Example 6

### Expression Times of the “Tespec PRO-1” and “Tespec PRO-2” Genes in the Testis

In mice, the primordial germ cells emerge in the fetus 7 days after fertilization, and they migrate to the genital ridge (11 days after fertilization) and differentiate into precursor cells of spermatogonium (13 days after fertilization). The precursor cells of spermatogonium enter into the arrested state from then on. They become spermatogonia, germ-line stem cells, after birth and then start their self-proliferation and differentiation into sperm. It takes about 34 days for spermatogonia to differentiate via spermatocytes and spermatids into mature sperm (in actuality, since spermatogonia per se have their own differentiation stage, if this stage is included, the period required for maturation is about 42 days in total). Then, testes of postnatal mice are collected per day after birth to verify the expression of “Tespec PRO-1” and “Tespec PRO-2”. This reveals at what stage of differentiation the genes are expressed in the sperm, or whether the genes are expressed in nurse cells (e.g. Sertoli's cells and Leydig's cells) in the testis.

On one hand, there exists a mutant mouse W (White spotting) that has a defect in chromosome 5 (Besmer, P. et al. (1993) Dev. Suppl., 125-137). This mutant mouse has a defect in c-kit, which is a receptor tyrosine kinase and expressed in the spermatogonia and spermatocytes. The mutant mouse has a deficiency in germ cells (complete deficiency) or a differentiation insufficiency (partial deficiency) at the stages after spermatogonium, though it has normal nurse cells such as Sertoli's cells and Leydig's cells in the testis. Thus, the expression of “Tespec PRO-1” and “Tespec PRO-2” were verified in the testis of the mutant mice W/Wv.

RT-PCR was performed by using, as templates, cDNAs prepared from total RNAs isolated from mouse testes 4 days, 8 days, 12 days, 18 days, and 42 days after birth, and from testes of three W/Wv mice 56 days after birth. In this RT-PCR experiment, cDNAs from adult mouse testis and liver were also used. Primers used were the “Tespec PRO-1”-specific primer and “Tespec PRO-2”-specific primer described above in Example 5. In the same manner as described in Example 5, 40 cycles (29 cycles for G3PDH) of PCR was conducted.

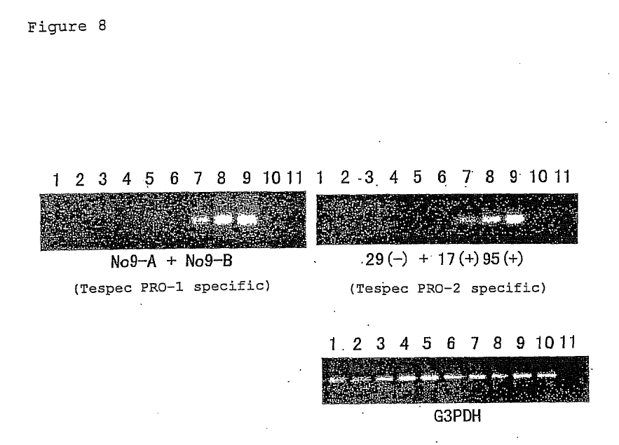

The result of RT-PCR demonstrate that expression levels of “Tespec PRO-1” and “Tespec PRO-2” were elevated in the testis 18 days after birth and later; neither gene was expressed at all before 12 days after birth nor in the testis of W/Wv mutant mouse (FIG. 8). No expression of the genes was detected in the liver, a negative control. These results suggest that both “Tespec PRO-1” and “Tespec PRO-2” are expressed not in the nurse cells such as Sertoli's cells and Leydig's cells, but in germ cells, and that their expression levels are elevated in the spermatocytes differentiated from germ cells or in the spermatids after meiosis.

### Example 7

### Cloning of Full-Length cDNA of Human “Tespec PRO-2”

Human “Tespec PRO-2” cDNA was cloned, based on the nucleotide sequence of mouse “Tespec PRO-2”. Human testis poly A+ RNA (CLONTECH) was converted into cDNA by using the reverse transcriptase SuperScript II (GIBCO) and (dT)₃₀VN primer. PCR was carried out, by using the cDNA as a template as well as using No9-G and No9-Q primers derived from mouse “Tespec PRO-2”. Polymerase used was AmpliTaq Gold and the thermal cycling profile of the low stringency PCR was: pre-heat at 95° C. for 12 minutes, 42 cycles of denaturation at 96° C. for 20 seconds, annealing at 55° C. for 20 seconds and extension at 72° C. for 30 seconds, and subsequent final extension at 72° C. for 3 minutes.

The resulting RT-PCR product was sequenced directly to determine the nucleotide sequence. The result showed that this PCR product is a gene fragment of human “Tespec PRO-2”, which exhibits about 80% homology to mouse “Tespec PRO-2” in nucleotide sequence. Based on this nucleotide sequence, primers for 5′RACE, i.e. h-B (SEQ ID NO: 31/5′-AGA GGT CAC TGT CGA GCT GGG-3′) and h-D (SEQ ID NO: 32/5′-TGT GAA TAA TGA CCT TCT GCA C-3′), and primers for 3′ RACE, i.e. h-A (SEQ ID NO: 33/5′-TTC AGC AAC ATC CAC TCG GAG A-3′) and h-C (SEQ ID NO: 34/5′-AAG CAA GTG CAG AAG GTC ATT A-3′) were generated. Nested 3′ and 5′ RACE was conduced by using human testis Marathon ready cDNA (CLONTECH) as a template, according to the manufacturer's instruction. As a result, a full-length cDNA for human “Tespec PRO-2” was cloned successfully. The nucleotide sequence is shown in SEQ ID NO: 5 and the amino acid sequence thereof is shown in SEQ ID NO: 6.

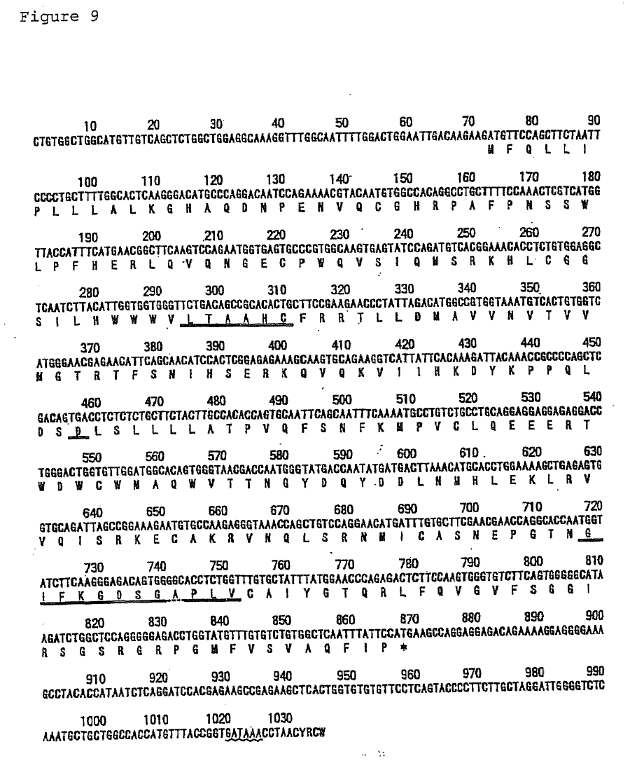

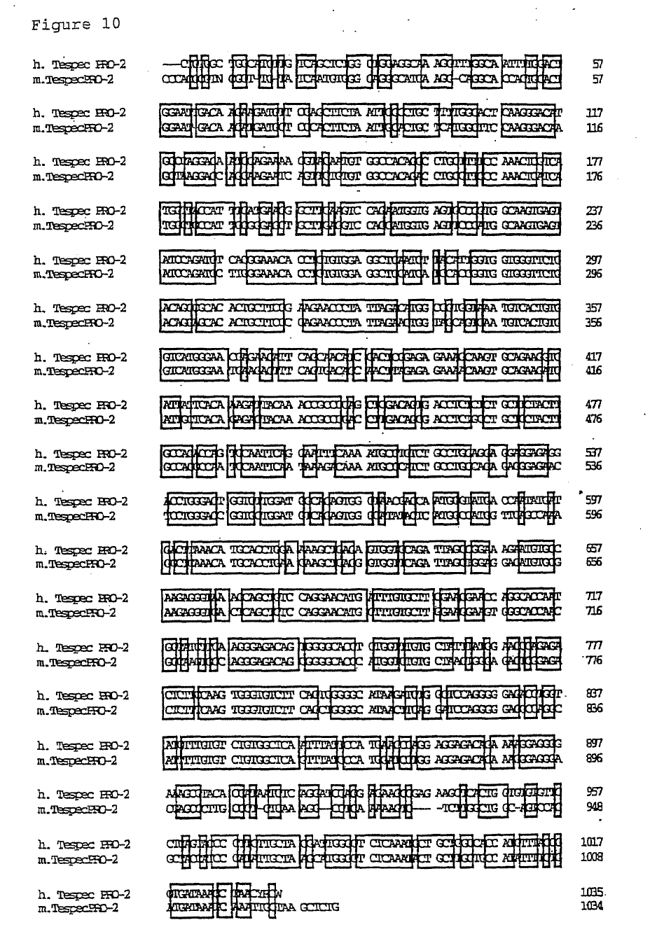

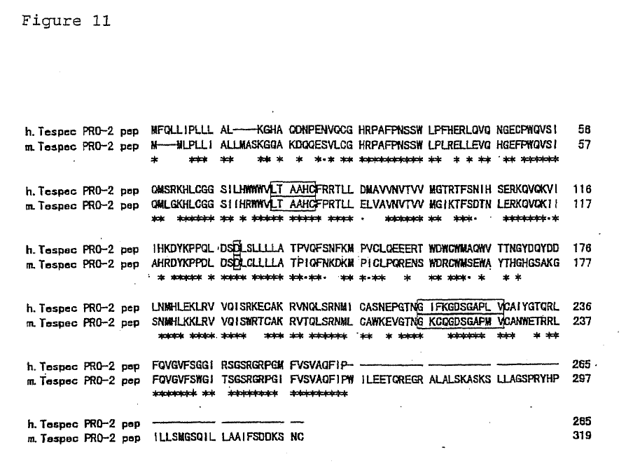

The human “Tespec PRO-2” cDNA consists of 1035 nucleotides and is predicted to encode 265 amino acids (FIG. 9). Homology between human and mouse “Tespec PRO-2” is 74.2% at the nucleotide level and 69.8% at the amino acid level. The amino acid sequence of the human “Tespec PRO-2” is shorter than that of mouse “Tespec PRO-2” by 54 residues at the C-terminus, and consequently, the human nucleotide sequence is longer in the 3′ non-coding region as compared with that of the mouse gene (FIGS. 10 and 11). In addition, there is a region predicted to be a signal peptide at the N-terminus, and the C-terminal region is also rich in hydrophobic amino acids. The deduced amino acid sequence of human “Tespec PRO-2” contains a trypsin-family serine protease motif, “Trypsin-His”. The motif of “Trypsin-Ser” of this protein contains an amino acid residue (GIFKGDSGAPLV) (SEQ ID NO:48) that is deviated from the consensus sequence in this motif that consists of 12 amino acid residues ([DNSTAGC]-[GSTAPIMVQH]-X-X-G-[DE]-S-G-[GS]-[SAPHV]-[LIVMFYWH]-[LIVMFYSTANQH]) (SEQ ID NO:47) (mouse “Tespec PRO-2” contains two amino acid residues deviated from the consensus sequence in this motif that consists of 12 amino acid residues).

The result of database search demonstrates that no known genes or proteins exhibit significant homology to the human “Tespec PRO-2”, at nucleotide and amino acid levels, revealing that this protein is a novel trypsin-family serine protease.

### Example 8

### Chromosomal Mapping of Human “Tespec PRO-2”

PCR was performed by using a human chromosome panel (CORRIELL CELL REPOSITORIES) as a template, a pair of primers, h-A and h-F (SEQ ID NO: 35/5′-CAT TGG TCG TTA CCC ACT GTG C-3′), and Advantage cDNA polymerase (CLONTECH) as polymerase. The thermal cycling profile of PCR was: pre-heat at 95° C. for 1 minute, 37 cycles of denaturation at 96° C. for 15 seconds, annealing at 60° C. for 15 seconds and extension at 68° C. for 30 seconds, and subsequent final extension at 68° C. for 3 minutes. The PCR reaction was subjected to electrophoresis on a 1.5% Seakem GTG agarose (TaKaRa).

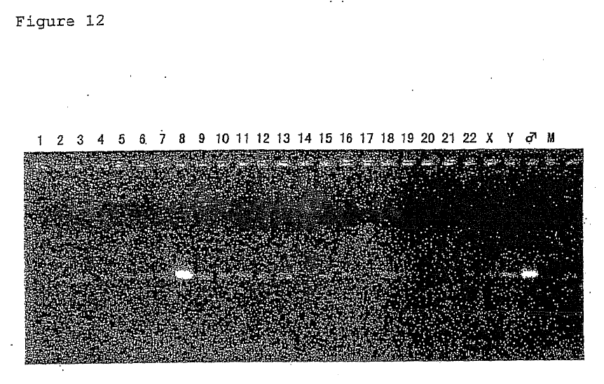

As the result of PCR, human “Tespec PRO-2” was mapped on chromosome 8 (FIG. 12).

### Example 9

### Cloning of Full-Length cDNA of the Human “Tespec PRO-3” Gene

Human testis poly A+ RNA (CLONTECH) was converted into cDNA by using the reverse transcriptase SuperScript II (GIBCO) and (dT)₃₀VN primer. RT-PCR was carried out by using the cDNA synthesized as a template, and the primer pair of PRO1-E (SEQ ID NO: 36/5′-ATT CTC AAT GAG TGG TGG GTT CT-3′) and PRO1-D (SEQ ID NO: 37/5′-CCA GCA CAC AGC ATA TTC TTG G-3′) that are synthesized on the basis of the nucleotide sequence of mouse “Tespec PRO-1”. The low stringency PCR was performed using the polymerase AmpliTaq Gold and the thermal cycling profile of: pre-heat at 95° C. for 12 minutes, 5 cycles of denaturation at 96° C. for 20 seconds, annealing at 50° C. for 20 seconds, and extension at 72° C. for 45 seconds, and subsequent 35 cycles of denaturation at 96° C. for 20 seconds, annealing at 60° C. for 20 seconds, and extension at 72° C. for 45 seconds, and final extension at 72° C. for 3 minutes.

The RT-PCR product was purified by gel filtration and then its nucleotide sequence was determined. The sequence analysis has revealed that this product is a gene fragment encoding a trypsin-family serine protease. The translation of this gene fragment revealed that it contained a “Trypsin-His” motif. A database search for the nucleotide sequence of this gene fragment showed that it overlaps in part with the sequence of a human EST (AA781356, aj25c04.s1 Soares-testis-NHT Homo sapiens cDNA clone 1391334 3′, mRNA sequence). Translation of this EST revealed the presence of a “Trypsin-Ser” motif in the amino acid sequence. Then, on the basis of the nucleotide sequence of the gene fragment obtained, primers were prepared: hPRO3-B (SEQ ID NO: 38/5′-GGA AAC AGC TCC TCG GAA TAT AAG C-3′) and hPRO3-D (SEQ ID NO: 39/5′-TGG ATG GGC TAG TTA AGT CGT TGG T-3′) for 5′RACE, and hPRO3-A (SEQ ID NO: 40/5′-TTC GAG GGA AGA ACT CGG TAT TC-3′) and hPRO3-C (SEQ ID NO: 41/5′-TGT GAA AAC GGA TCT GAT GAA AGC G-3′) for 3′ RACE. Nested RACE was conducted by using human testis Marathon ready cDNA (CLONTECH) as a template, according to the manufacturer's instruction to clone a full-length cDNA. The product obtained by the RACE was sequenced directly or after subcloned into the pGEM T easy vector. The nucleotide sequence is shown in SEQ ID NO: 9 and the amino acid sequence is shown in SEQ ID NO: 10.

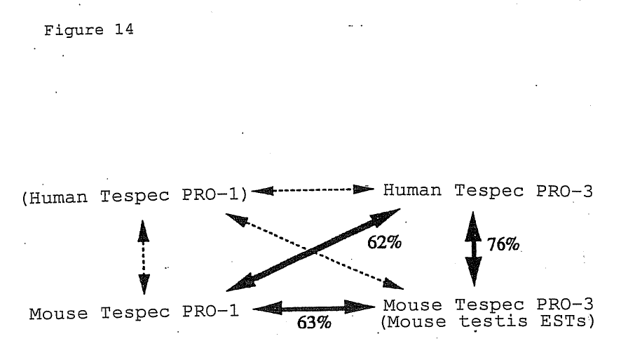

This novel human gene showed higher homology to mouse testis ESTs deposited in the database (AA497965, AA497934, AA497919, etc.) than to mouse “Tespec PRO-1” (FIG. 14), though this gene was obtained using the primers generated on the basis of the nucleotide sequence of mouse “Tespec PRO-1”. Thus, the gene was designated human “Tespec PRO-3”.

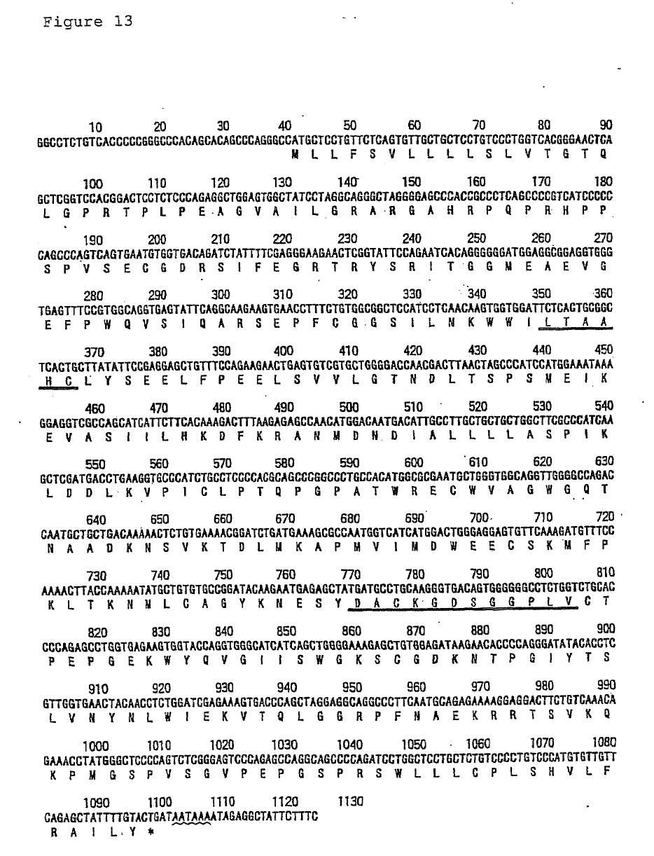

The human “Tespec PRO-3” cDNA consists of 1123 nucleotides and is predicted to encode 352 amino acids (FIG. 13). This gene has a putative signal peptide at its N-terminus, and contains the “Trypsin His” and “Trypsin-Ser” motifs. In addition, cysteine residues that are predicted to form an intramolecular a disulfide bond are well conserved relative to other serine proteases.

### Example 10

### Cloning of Full-Length cDNA of the Mouse “Tespec PRO-3” Gene

Mouse “Tespec PRO-3”, which is the mouse counterpart of the above-mentioned human “Tespec PRO-3” is considered to contain some of the nucleotide sequences of the above-mentioned ESTs, which are derived from mouse testis. Mouse ESTs for this gene, eight sequences in total, have been deposited in a database. Among them, four ESTs are derived from the testis, one is derived from the kidney and the remaining three are derived from cDNAs of unknown origins. Thus, primers were designed on the basis of these ESTs to conduct RACE using mouse testis Marathon ready cDNA as a template, and the full-length cDNA sequence of mouse “Tespec PRO-3” was cloned.

On the basis of the nucleotide sequences of the mouse ESTs (AA497965, AA497934, AA497919, AA497949, AA271404, AA238183, AA240375, and AA105229), primers for 5′ RACE, i.e. mPRO3-B (SEQ ID NO: 42/5′-CAC CTA CTG CCA GGA TCT GTG G-3′) and mPRO3-D (SEQ ID NO: 43/5′-GGC TAT TTT CTC AAT CCA CAG GGT A-3′), and primers for 3′ RACE, i.e. mPRO3-A (SEQ ID NO: 44/5′-ATA GAG TGG GAG GAA TGC TTA CAG A-3′) and mPRO3-C (SEQ ID NO: 45/5′-GCT ACG ATG CTT GCC AGG GTG-3′), were generated. Nested RACE was conducted by using the mouse testis Marathon ready cDNA (CLONTECH) as a template, according to the manufacturer's instruction. The product obtained by RACE was sequenced directly or after subcloned into the pGEM T easy vector. The nucleotide sequence is shown in SEQ ID NO: 7 and the amino acid sequence is shown in SEQ ID NO: 8.

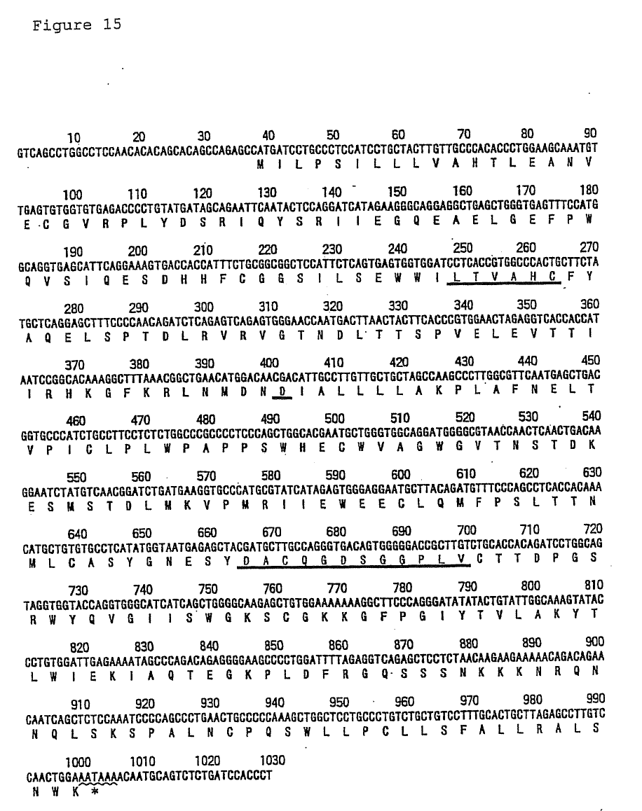

The mouse “Tespec PRO-3” cDNA consists of 1028 nucleotides and it is predicted to encode 321 amino acids (FIG. 15). While the deduced amino acid sequence contains a “Trypsin-Ser” motif, it has the “Trypsin-His” motif that is deviated from the consensus motif consisting of 6 amino acids [LIVM]-[ST]-A-[STAG]-H-C (SEQ ID NO:49) at one amino acid residue (LTVAHC) (SEQ ID NO:50). However, like mouse “Tespec PRO-2”, some known trypsin-family serine proteases have sequences containing several amino acid deviation in the consensus sequence, and therefore mouse “Tespec PRO-3” is predicted to function as a protease. In addition, it has a hydrophobic region predicted to be a signal peptide at its N-terminus. Cysteine residues predicted to form an intramolecular disulfide bond are well conserved in the sequence relative to other serine proteases.

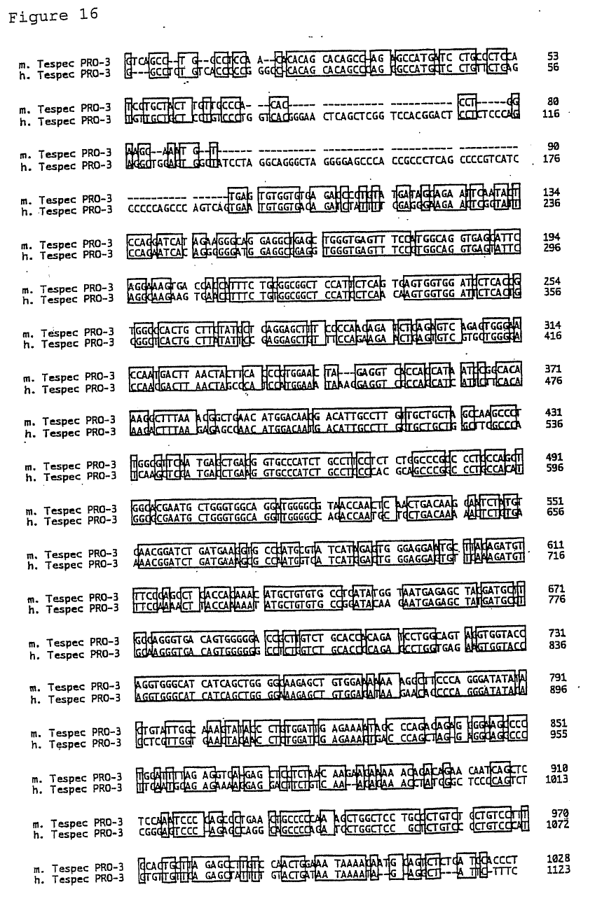

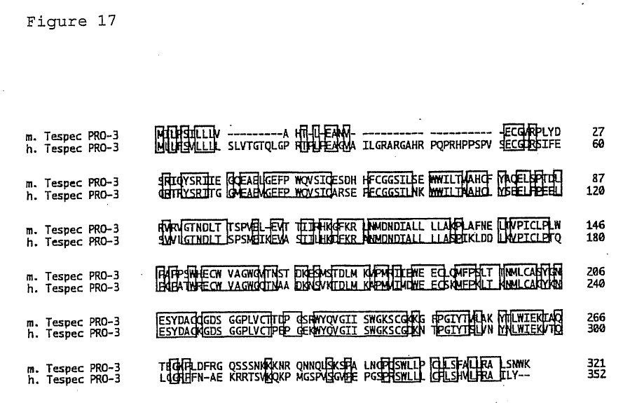

Homology between human and mouse “Tespec PRO-3” is 70.2% at the nucleotide level and 59.6% at the amino acid level (FIGS. 16 and 17). It was revealed that compared to human “Tespec PRO-3”, mouse “Tespec PRO-3” is shorter in nucleotide sequence by about 100 residues at the 5′ end, and also shorter in amino acid sequence by about 30 residues at the N-terminus.

### INDUSTRIAL APPLICABILITY

Provided by the present invention are novel trypsin-family serine proteases and the genes encoding them. The proteins of the present invention were suggested to be involved in sperm differentiation and maturation or in sperm function (fertilization). Thus, the proteases of the present invention and the genes thereof are expected to serve for developing new therapeutic or diagnostic agents for infertility and for developing new contraceptives.

What is claimed is:

1. An isolated protein comprising an amino acid sequence selected from the group consisting of SEQ ID NO: 4, SEQ ID NO: 6, SEQ ID NO: 8, and SEQ ID NO: 10.

2. An isolated protein comprising an amino acid sequence selected from the group consisting of SEQ ID NO: 4, SEQ ID NO: 6, SEQ ID NO: 8, and SEQ ID NO: 10, wherein up to 30 amino acids are deleted, added, inserted and/or substituted with different amino acids, wherein said protein has protease activity.

3. A partial peptide of the protein according to claim 1 or 2.

4. A fusion protein comprising the protein according to claim 1 or 2, fused with another peptide.

5. An isolated DNA selected from the group consisting of:
(a) a DNA comprising a nucleotide sequence selected from the group consisting of SEQ ID NO: 3, SEQ ID NO: 5, SEQ ID NO: 7, and SEQ ID NO: 9;
(b) a DNA encoding a protein comprising an amino acid sequence selected from the group consisting of SEQ ID NO: 4, SEQ ID NO: 6, SEQ ID NO: 8, and SEQ ID NO: 10;
(c) a DNA encoding a protein comprising an amino acid sequence selected from the group consisting of SEQ ID NO: 4, SEQ ID NO: 6, SEQ ID NO: 8, and SEQ ID NO: 10, wherein up to 30 amino acids are deleted, added, inserted and/or substituted with different amino acids, wherein said protein has protease activity; and
(d) a DNA which hybridizes under the stringent conditions of 42° C., 2×SSC, 0.1% SDS to the complement of a DNA comprising a nucleotide sequence selected from the group consisting of SEQ ID NO: 3, SEQ ID NO: 5, SEQ ID NO: 7, and SEQ ID NO: 9, wherein said protein has protease activity.

6. A vector comprising the DNA of claim 5.

7. A transformed cell comprising the DNA according to claim 5 in an expressible form.

8. A method for producing the protein according to claim 1 or 2, said method comprising the steps of: culturing the transformed cell according to claim 7, and recovering the expressed protein from the transformed cell or the culture supernatant thereof.

9. A method of screening for a substrate of the protein according to claim 1 or 2, said method comprising the following steps of:
(a) contacting a test sample with said protein;
(b) detecting the protease activity of said protein against the test sample; and
(c) selecting a compound that is digested or cleaved by said protease activity.

10. A substrate of the protein according to claim 1 or 2, wherein said substrate can be isolated by the method according to claim 9.

11. A method of screening for a compound capable of inhibiting the activity of the protein according to claim 1 or 2, said method comprising the following steps of:
(a) contacting the protein with the substrate identified by the method of claim 9 in the presence of a test sample;
(b) detecting the protease activity of the protein against the substrate; and
(c) selecting a compound that reduces the protease activity relative to the protease activity detected in the absence of the test sample.

12. A compound that inhibits the activity of the protein according to claim 1 or 2, wherein said compound can be isolated by the method according to claim 11.

13. An antibody that binds to the protein according to claim 1 or 2.

14. A method for detecting or assaying the protein according to claim 1 or 2, said method comprising the steps of: contacting the antibody according to claim 13 with a test sample that is anticipated to contain the protein; and detecting or assaying formation of the immune-complex between the antibody and the protein.

15. A nucleotide sequence specifically hybridizing to the DNA comprising a nucleotide sequence selected from the group consisting of SEQ ID NO: 3, SEQ ID NO: 5, SEQ ID NO: 7, and SEQ ID NO: 9, wherein the nucleotide sequence is at least 15 nucleotides in length.
